(19) United States
(12) Patent Application Publication
(10) Pub. No.: US 2022/0370075 A1
(43) Pub. Date: Nov. 24, 2022

(54) CLIP UNIT AND CLIP DEVICE

(71) Applicant: OLYMPUS MEDICAL SYSTEMS CORP., Tokyo (JP)

(72) Inventors: Toshinori TAMURA, Hirosaki-shi (JP); Kensuke UESAKA, Hino-shi (JP)

(73) Assignee: OLYMPUS MEDICAL SYSTEMS CORP., Tokyo (JP)

(21) Appl. No.: 17/749,381

(22) Filed: May 20, 2022

Related U.S. Application Data
(60) Provisional application No. 63/191,389, filed on May 21, 2021.

(51) Int. Cl. A61B 17/128

(52) U.S. Cl. CPC A61B 17/1285

(57) ABSTRACT

Clip unit with a clip having a plurality of arms that can be opened and closed, a distal end connecting portion that is releasably connected to the clip, a proximal end connecting portion that is connected to a power transmission member; and a rod-shaped portion that longitudinally connects the distal end connecting portion and the proximal end connecting portion. In a cross-section perpendicular to the longitudinal direction, the rod-shaped portion has a first length in a first direction perpendicular to the longitudinal direction that is shorter than a second length in a second direction perpendicular to both the longitudinal direction and the first direction.

### RELATED APPLICATION DATA

This application is based on and claims priority under 35 U.S.C. § 119 to U.S. Provisional Application No. 63/191,389 filed on May 21, 2021, the entire contents of which are incorporated herein by reference.

### FIELD OF DISCLOSURE

The present disclosure relates to a clip unit for an endoscope and a clip device.

### BACKGROUND

In endoscopic treatment, a clip unit is used that is capable of ligating an excised portion of tissue or the like after a treatment to perform hemostasis or the like. The clip unit includes a clip for sandwiching a cutting portion of tissue or the like, a holding tube for accommodating the clip and locking the clip in a closed state, and the like. The clip unit is introduced into the treatment position by an introduction device capable of being inserted through a channel of an endoscope as shown in JP Pat. No. 5,750,620. However, when the clip unit is inserted through the channel of the endoscope by the introducing device, the clip unit may not easily pass through the curved channel of the endoscope.

### SUMMARY

In view of the above circumstances, it is an object of the present invention to provide a clip unit and a clip device which are easily passed through a channel of a curved endoscope, while also limiting breakage of the connecting member that connects the clip to the power transmission member.

In order to solve the above problems, the present invention proposes the following means.

A clip unit according to a first aspect of the present invention comprises: a clip having a plurality of arms configured to be opened and closed; a connecting member including a rod-shaped portion having a longitudinal axis extending in a longitudinal direction and that longitudinally connects a distal end connecting portion and a proximal connecting portion; and a power transmission member, where the distal end connecting portion is releasably connected to the clip and the proximal connecting portion is connected to the power transmission member, and where the rod-shaped portion, in a cross-section perpendicular to the longitudinal axis, has a first length in a first direction perpendicular to the longitudinal axis and a second length in a second direction perpendicular to the longitudinal axis, where the first direction and the second direction are also perpendicular to each other and the first length is shorter than the second length.

A clip device according to a second aspect of the present invention includes: a clip having a plurality of arms configured to be opened and closed; a power transmission member that operates the clip; a connecting member including a distal connecting portion that is releasably connected to the clip, a proximal connecting portion that is connected to the power transmission member, and a rod-shaped portion having a longitudinal axis extending in a longitudinal direction and that connects the distal connecting portion and the proximal connecting portion in a longitudinal direction; and a sheath through which the clip, the power transmission member, and the connecting member pass through, and where the rod-shaped portion, in a cross-section perpendicular to the longitudinal axis, has a first length in a first direction perpendicular to the longitudinal axis and a second length in a second direction perpendicular to the longitudinal axis, where the first direction and the second direction are also perpendicular to each other and the first length is shorter than the second length.

In certain exemplary embodiments, the rod-shaped portion, in a cross-section perpendicular to the longitudinal axis, has a shape of an ellipse in which the first direction corresponds to the minor axis of the ellipse and the second direction corresponds to the major axis of the ellipse. In alternative embodiments, the rod-shaped portion, in a cross-section perpendicular to the longitudinal axis, has a shape of an ovid, a crescent, a triangle, a rectangle, a trapezoid, or a polygon having 5 or more sides, such as between 5 and 20 sides. Regular and irregular polygons can be used.

The clip unit and clip device of the present invention are easy to pass through a channel of a curved endoscope.

Additional features and advantages will be set forth in the description that follows, and in part will be apparent from the description, or may be learned by practice of the disclosure. The objectives and other advantages disclosed herein will be realized and attained by the structure particularly pointed out in the written description and claims thereof, as well as the appended drawings.

It is to be understood that both the foregoing general description and the following detailed description are exemplary and explanatory and are intended to provide further explanation of the disclosure as claimed.

### BRIEF DESCRIPTION OF THE DRAWINGS

The accompanying drawings, which are included to provide a further understanding of the disclosure and are incorporated in and constitute a part of this application, illustrate embodiments of the disclosure and together with the description serve to explain principles of the disclosure. It should be understood that the embodiments depicted are not limited to the precise arrangements and instrumentalities shown.

FIG. 1 is a perspective view of a clip introduction device.

FIG. 2 is a perspective view of a clip unit according to a first embodiment of the present invention.

FIG. 3 is a perspective view of the clip unit and showing features internal to the holding tube.

FIG. 4 is a plan view of a connecting member of the clip unit.

FIG. 5 is a cross-sectional view taken along X-X shown in FIG. 4.

FIG. 6 is a cross-sectional view of the clip unit inserted into the sheath.

FIG. 7 is a perspective view of a cartridge containing the clip unit.

FIG. 8 is a cross-sectional view of a cartridge containing the clip unit.

FIG. 9 is a cross-sectional, exploded view of the same cartridge with the lid removed.

FIG. 10 is a perspective view of a regulating member of the cartridge.

FIG. 11 is a perspective view in which the regulating member is gripped by a pair of arms of the clip.

FIG. 12 is a plan view of the regulating member.

FIG. 13 is a plan view in which the regulating member is gripped in the first area.

FIG. 14 is a diagram illustrating a method of loading a clip unit using the same cartridge.

FIG. 15 is a diagram illustrating a method of loading a clip unit using the same cartridge.

FIG. 16 is a diagram illustrating a method of loading a clip unit using the same cartridge.

FIG. 17 is a diagram illustrating a method of loading a clip unit using the same cartridge.

FIG. 18 is a diagram illustrating a method of loading a clip unit using the same cartridge.

FIG. 19 is a diagram illustrating a method of loading a clip unit using the same cartridge.

FIG. 20 is a diagram illustrating a method of loading a clip unit using the same cartridge.

FIG. 21 is a diagram illustrating a method of loading a clip unit using the same cartridge.

FIG. 22 is an enlarged view of a holding pipe shown in FIG. 21.

FIG. 23 is a diagram illustrating a method of loading a clip unit using the same cartridge.

FIG. 24 is a diagram illustrating a method of loading a clip unit using the same cartridge.

FIG. 25 is a diagram illustrating a method of loading a clip unit using the same cartridge.

FIG. 26 is a diagram illustrating a method of loading a clip unit using the same cartridge.

FIG. 27 is a diagram showing the clip unit in a state as would be after introduction into a body.

FIG. 28 is a diagram showing the clip unit having a pair of arms closed.

FIG. 29 shows the clip unit with the clip locked.

FIG. 30 is a diagram showing the clip unit in which the clip is separated from the connecting member.

FIG. 31 is a diagram showing the clip unit after breaking from the sheath.

FIG. 32 is a sectional view of an alternative embodiment of a retractable wing.

FIG. 33 is a cross-sectional view showing the alternative embodiment of the retractable wing of FIG. 32 in a portion of the pressing tube.

FIG. 34 is a perspective view of a section of a pressing tube having retractable wings.

FIG. 35 is a side view of the section of a pressing tube having retractable wings shown in FIG. 34.

FIGS. 36A, 36B and 36C are an end view, a first cross-section view, and a second cross-sectional view, respectively, of the male part of the section of a pressing tube shown in FIG. 35.

FIGS. 37A and 37B show the section with the retractable wings aligned with (FIG. 37A) and pivoted relative to (FIG. 37B) the section with the pressing tube body.

FIG. 38 is a perspective view of a connecting member in a pressing tube 3 and shows an embodiment of an alignment structure.

FIG. 39 is cross-sectional view taken along section F-F in FIG. 38.

FIGS. 40A to 40G schematically illustrate cross-sections of alternative embodiments of the alignment structure.

Throughout all of the drawings, dimensions of respective constituent elements are appropriately adjusted for clarity. For ease of viewing, in some instances only some of the named features in the figures are labeled with reference numerals.

### DETAILED DESCRIPTION

Reference will now be made in detail to embodiments of the present disclosure, examples of which are illustrated in the accompanying drawings.

### First Embodiment

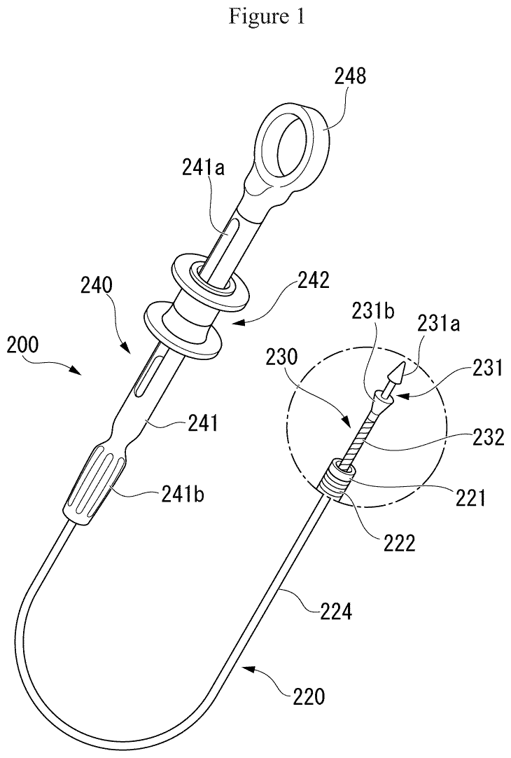

A first embodiment of the present invention will be described with reference to FIGS. 1 to 31. The cartridge system 100 includes a clip unit 1 and a cartridge 5 that accommodates the clip unit 1. The cartridge system 100 is a support system for easily loading the clip unit 1 into the clip applicator 200.

Clip Applicator

FIG. 1 is a perspective view of a clip applicator 200. The clip applicator 200 includes a sheath 220, an operation wire 230, and an operation unit 240. The clip applicator 200 is inserted into a treatment instrument insertion channel of an endoscope, for example, and is used in combination with an endoscope. Therefore, the sheath 220 is formed sufficiently longer than the length of the treatment instrument insertion channel of the endoscope. The sheath 220 is flexible and curves to conform to the curvature of the insertion portion of the endoscope.

Sheath 220 includes a tip 221, a distal coil 222, and a proximal coil 224, and is formed, generally, in an elongated tubular shape. The distal coil 222 is disposed on the distal end of the sheath 220. Tip 221 is disposed at the distal end side of the distal coil 222.

Operation wire 230 (also called a power transmission member), as shown in FIG. 1, has an arrowhead hook portion 231 (also called a connecting portion) configured to releasably connect to the clip unit 1 and a wire 232 for operating the arrowhead hook portion 231. The arrowhead hook portion 231 includes an engagement portion 231a having a substantially conical shape that engages with the clip unit 1, and a wire connection portion 231b provided at the proximal end of the engagement portion 231a.

Arrowhead hook portion 231 is formed of a metal material such as, for example, stainless steel. The wire 232 is located in the channel of the sheath 220 so as to freely move forward and backward. The distal end of the wire 232 is fixed to the proximal end of the wire connecting portion 231b, for example by welding.

As shown in FIG. 1, the operation unit 240 includes an operation unit main body 241, a slider 242, and a thumb ring 248. The operation unit main body 241 is injection molded of, for example, a resin material. The operation unit main body 241 includes a slit portion 241a and a rotary grip 241b on the distal end side. The slit portion 241a supports the slider 242 so as to be able to move the slider 242 forward and backward along the length of the slit portion 241a. Slider 242 is retractably mounted in the longitudinal axis direction of the operation unit body 241 and the proximal end of the wire 232 is attached to the slider 242. As the slider 242 moves forward and backward along the operation unit main body 241, the wire 232 moves forward and backward with respect to the sheath 220, and the arrowhead hook portion 231 moves forward and backward. Thumb ring 248 is located at the proximal end of the operation unit body 241 and is rotatably mounted about the longitudinal axis of the operation unit body 241.

Clip Unit

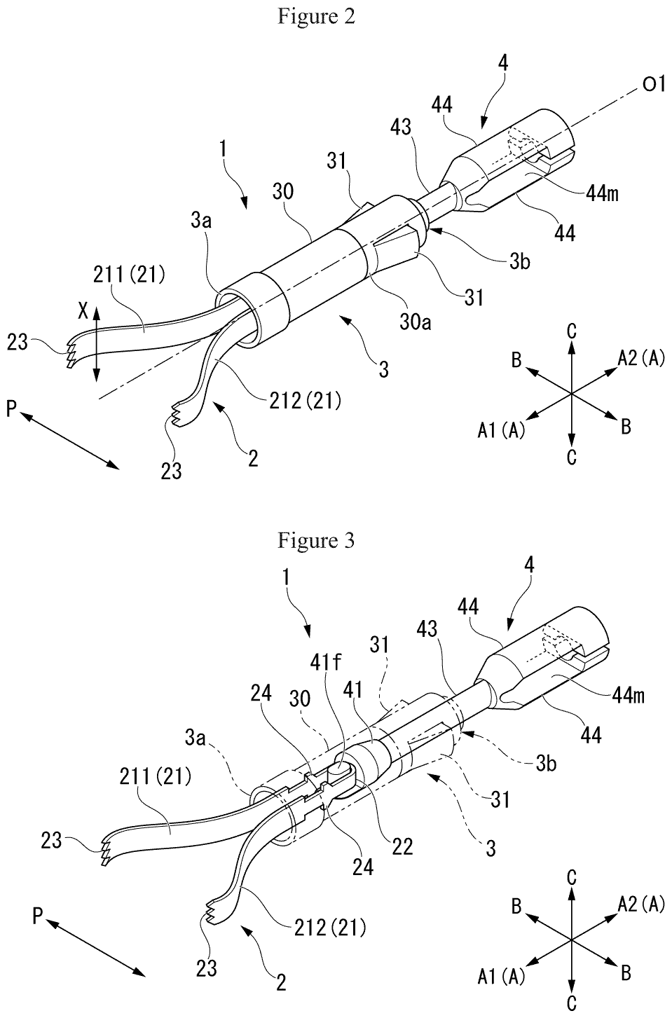

FIG. 2 is a perspective view of the clip unit 1 according to the present embodiment. Clip unit 1 includes a clip 2, a pressing tube 3 as a tightening member, and a connecting member 4. FIG. 3 is a perspective view of the clip unit 1 in which the pressing tube 3 is displayed in a transparent manner so as to show features internal to the pressing tube 3.

In the following description, the side of the clip 2 in the longitudinal direction A of the clip unit 1 is referred to as a tip side A1 (or a distal side) of the clip unit, and the connecting member 4 side is referred to as a proximal side A2 of the clip unit 1. A direction perpendicular to the longitudinal direction A is defined as a “first direction B” or a “left-right direction B”. A direction perpendicular to the longitudinal direction A and the first direction B is defined as a “second direction C” or a “vertical direction C”. The directions A, B, and C are shown in the axes accompanying the figures and which illustrate the directions A, B, and C as being orthogonal.

Clip 2 is formed by bending a metal plate material such as a plate spring material, for example, stainless steel material, at the center portion. Clip 2 has a pair of arms 21 which are configured to be opened and closed, and a connecting portion 22 for connecting the pair of arms 21. A pair of arms 21 has a first arm 211 and a second arm 212. The first arm 211 and the second arm 212 are disposed symmetrically with respect to the central axis O1 in the longitudinal direction A of the clip unit 1. Tissue grasping portions 23 are formed at the distal ends of the pair of arms 21 and face each other. The tissue grasping portions 23 are formed by bending the distal ends of the pair of arms 21 inward.

Toward the proximal end of the pair of arms 21, the pair of arms 21 include an engaging portion 24 protruding in a direction perpendicular to the longitudinal direction A. The side of the engaging portion 24 located towards the tissue grasping portion 23 has a sharp slope forming an angle of 90 degrees or less relative to the direction of the central axis O1, and the side of the engaging portion 24 located towards the connecting portion 22 has an obtuse slope forming an obtuse angle relative to the direction of the central axis O1.

Connecting portion 22 is formed in a U-shape, typically by bending, and is connected to the connecting member 4 (as further described herein). Connecting portion 22 is biased so that the pair of arms 21 is in the open state. Therefore, the pair of arms 21 of the clip 2 has a self-expanding force with respect to the opening and closing direction P. When the connecting portion 22 is connected to the connecting member 4, the opening and closing direction P of the pair of arms 21 substantially coincides with the first direction B.

Pressing tube 3 has a pressing tube body 30 formed in a cylindrical shape, and retractable wings 31. The pressing tube body 30 is formed by injection molding a highly rigid resin material having a soft material than that of the clip 2, for example, PPA (polyphthalamide), PA (polyamide), or the like, with appropriate elasticity. In addition, the pressing tube body 30 may be formed of metal instead of a high-rigidity resin material.

Retractable wings (engaging portion) 31 include a pair of portions projecting from the outer peripheral surface 30a of the pressing tube body 30 and are typically convex to correspond with the shape of the outer peripheral surface 30a. A retractable wing 31 is provided on both sides of the pressing tube body 30 across the central axis O1. Retractable wing 31 can move between a protruding state and a retracted state. In a protruding state, the body of the retractable wings 31 protrudes outward in the radial direction with respect to the outer peripheral surface 30a of the pressing tube body 30; in the retracted state, the body of the retractable wings 31 are located within the envelope of the pressing tube body 30. The retractable wings 31 are made of an elastically deformable plastic and the retractable wings 31 are moved to the retracted stated by receiving an inward force in the radial direction. When the above-mentioned inward force is released, the retractable wings 31 move from the retracted state to the protruding state.

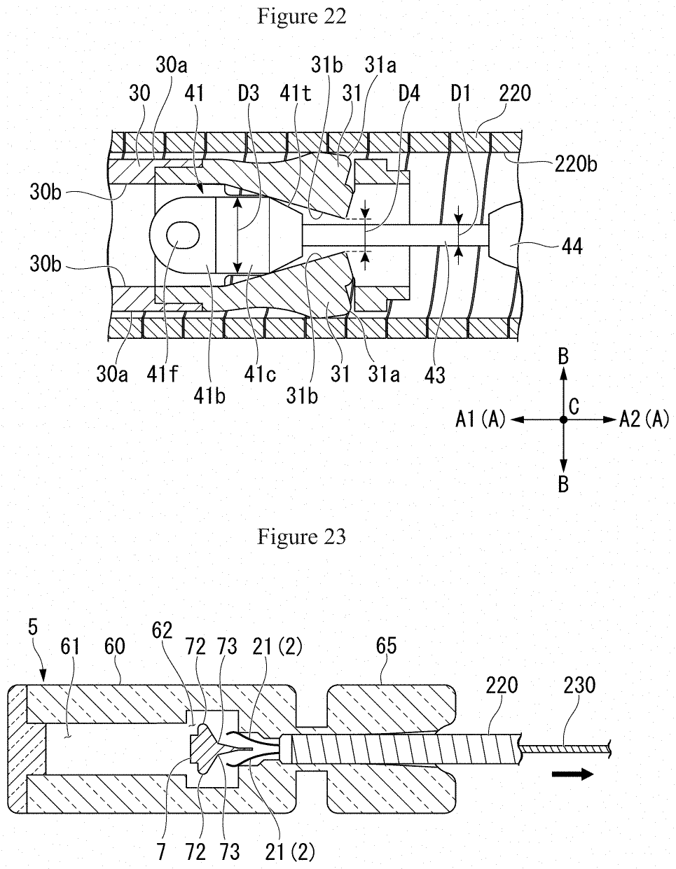

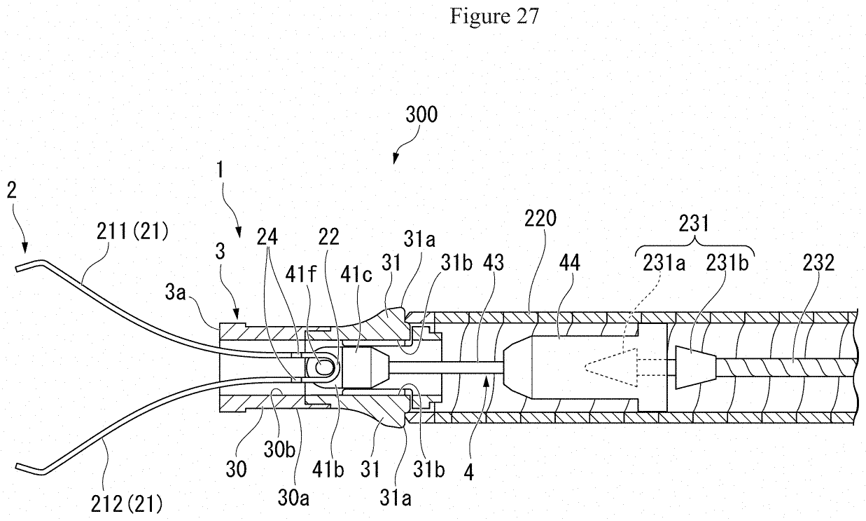

Preferably, when the retractable wing 31 is in a projecting state in the radial direction (see FIG. 27), the outer peripheral surface 31a of the retractable wing 31 protrudes outward from the outer peripheral surface 30a of the pressing tube body 30 and the inner peripheral surface 31b of the retractable wing 31 is in a position substantially equal to the inner peripheral surface 30b of the pressing tube body 30. Further, preferably, when the retractable wing 31 is in the retracted state in the radial direction (see FIG. 22), the outer peripheral surface 31a of the retractable wing 31 is substantially equal to the outer peripheral surface 30a of the pressing pipe body 30, or is at a position substantially equal to the inner peripheral surface 220b of the sheath 220, and the inner peripheral surface 31b of the retractable wing 31 is projecting inward relatively to the inner peripheral surface 30b of the pressing pipe body 30.

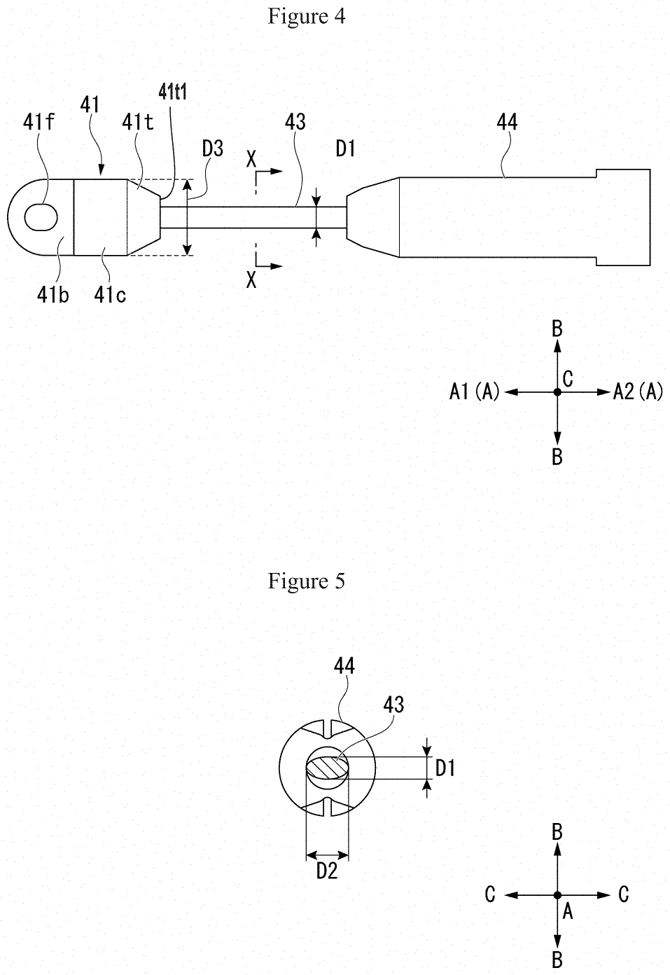

FIG. 4 is a plan view of the connecting member 4. The connecting member 4 extends in a longitudinal direction along a longitudinal axis and at one end is connected to the connecting portion 22 of the clip 2 and at the other end is connected to the operation wire 230 inserted through the sheath 220, such as by connection to arrowhead hook portion 231. That is, the connecting member 4 connects the clip 2 and the operation wire 230. Connecting member 4 includes a rod-shaped portion 43, a distal end connecting portion 41, which is inserted into the inner space of the pressing tube 3, and a proximal end connecting portion 44. The rod-shaped portion 43 longitudinally connects the proximal end of the distal end connecting portion 41 to the distal end of the proximal end connecting portion 44 along the longitudinal axis.

The distal end connecting portion 41 is an engaging portion engaged with the connecting portion 22 of the clip 2. Tip connecting portion 41 has a hook 41f provided on the distal end side A1, a breakage portion 41b provided on the proximal end side A2 of the hook 41f, and a cylindrical portion 41c provided on the proximal end side A2 of the breakage portion 41b. Hook 41f is a hook extending in a second direction C perpendicular to the longitudinal direction A and is formed in a substantially cylindrical rod shape. However, the shape of the hook 41f is not limited to a substantially cylindrical rod shape and other suitable shapes can be used. For example, shapes having an aspect ratio with the length in the perpendicular C-direction is greater than the length in the perpendicular direction B and/or the longitudinal direction A can be used. Such shapes include those that have, in a cross-section perpendicular to the perpendicular C-direction, a shape of: an ellipse, an ovid, a crescent, a triangle, a rectangle, a trapezoid, or a polygon having 5 or more sides, such as between 5 and 20 sides. Regular and irregular polygons can be used. The connecting portion 22 of the clip 2 is engaged on the hook 41f by, for example, hooking the connecting portion 22 on the hook 41 by inserting the hook 41f into the interior region of the bent center portion of the clip 2.

Breakage portion 41b is broken when the amount of breaking force, i.e., tensile force, applied to the proximal end side of the breakage portion by pulling connecting portion 22 relative to the hook 41f exceeds a predetermined amount, such as up to 90N (Newton), for example 20N. Optionally, the breakage portion 41b may have a mechanism for releasing the connection between the hook 41f of the connecting portion 22 and the connecting member 4 of the clip 2 with or without physical separation of the breakage portion 41b. For example, a mechanism for releasing the connection between the connecting portion 22 and the hook 41f can include deforming (without breaking) the breakage portion 41b by plastic deformation or elastic deformation, whereby the connecting portion 22 no longer engages with the hook 41f.

Cylindrical portion 41c is formed in a cylindrical shape. At the proximal end side A2 of the cylindrical portion 41c, a tapered surface 41t has a radial length that becomes smaller toward the proximal end side as the longitudinal position moves proximally along the longitudinal axis.

The rod-shaped portion 43 is a substantially rod-shaped member extending in the longitudinal direction A. The rod-shaped portion 43 is provided between the distal end connecting portion 41 and the proximal end connecting portion 44, and connects the distal end connecting portion 41 and the proximal end connecting portion 44. Typically, the length of the rod-shaped portion 43 in the longitudinal direction A is greater than the length of rod-shaped portion 43 in the perpendicular direction B and/or C. However, the shape of the rod-shaped portion 43 is not limited to a substantially cylindrical rod shape and other suitable shapes can be used that provide an aspect ratio (in cross-section perpendicular to the longitudinal direction A) that allows the rod-shaped portion 43 to bend in a desired direction. For example, shapes having (in a cross-section perpendicular to the longitudinal direction A) an aspect ratio with the length in the perpendicular direction B is greater than the length in the perpendicular direction C can be used and allow the rod-shaped portion 43 to more easily bend in the perpendicular direction C. Similarly, shapes having (in a cross-section perpendicular to the longitudinal direction A) an aspect ratio with the length in the perpendicular direction C is greater than the length in the perpendicular direction B can be used and allow the rod-shaped portion 43 to more easily bend in the perpendicular direction B. Such shapes include those that have, in a cross-section perpendicular to the longitudinal axis, a shape of: an ellipse, an ovid, a crescent, a triangle, a rectangle, a trapezoid, or a polygon having 5 or more sides, such as between 5 and 20 sides. Regular and irregular polygons can be used.

FIG. 5 is a sectional view taken along the X-X-line shown in FIG. 4. Rod-shaped portion 43, in a cross-section perpendicular to the longitudinal direction A, has a first length D1 in the first direction B that is shorter than the second length D2 in the second direction C (D2>D1). Rod-shaped portion 43, in a cross-section perpendicular to the longitudinal direction A, has a first length D1 is a shorter rectangular shape or elliptical shape than the second length D2. Other regular and irregular geometric shapes can be used, as described herein. Also and as shown in FIG. 4, the first length D1 of the rod-shaped portion 43 is shorter than the third length D3 in the first direction B of the distal end connecting portion 41.

Proximal end connecting portion 44 is configured to engage an engagement portion of the clip applicator 200, such as arrowhead hook portion 231. The proximal end connecting portion 44 is provided at the proximal end of the rod-shaped portion 43, and branches into a bifurcated shape. Proximal end connecting portion 44 is elastically deformable with respect to the rod-shaped portion 43 and the proximal end connecting portion 44 can be opened and closed with respect to the rod-shaped portion 43. A notch 44m for holding and accommodating the engagement portion 231a of the arrowhead hook portion 231 is formed between regions of the proximal end connecting portion 44. The notch 44m is formed in a shape that closely contacts the outer peripheral surface of the engagement portion 231a of the arrowhead hook portion 231.

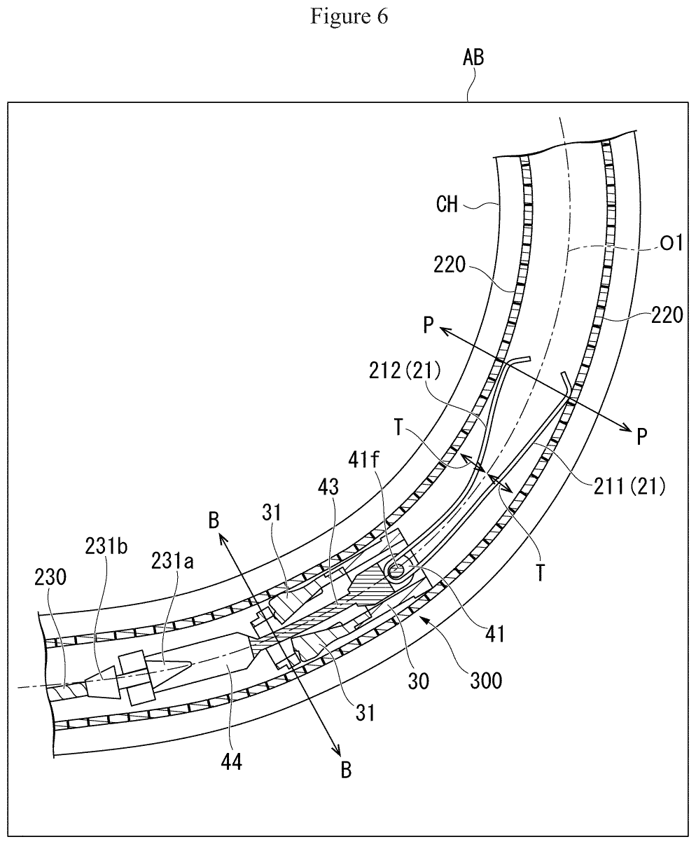

FIG. 6 is a cross-sectional, cut-away view of the clip unit 1 loaded into the sheath 220. The clip unit 1 loaded in the sheath 220 passes through the treatment instrument insertion channel CH of the curved endoscope and is introduced into the treatment position. The connecting member 4 has a rod-shaped portion 43 in which the first length D1 in the first direction B is shorter than the second length D2 in the second direction C, and is more easily curved in the first direction B than in the second direction C. The sheath 220 and the clip unit 1 loaded in the sheath 220 are also referred to as a clip device 300.

Opening and closing direction P of the pair of arms 21 of the clip 2 connected to the connecting member 4 substantially coincides with the first direction B. Since the pair of arms 21 is easily curved with respect to the opening/closing direction P, the clip 2 is also easily curved in the first direction B. Incidentally, if the clip unit 1 is curved as shown in FIG. 6, although the opening and closing direction P and the first direction B do not coincide perfectly, a plane including the opening and closing direction P and the first direction B and the horizontal central axis O1 (hereinafter, referred to as “plane AB”) may exist. In this case, the clip 2 tends to bend in the plane AB.

Each of the arms 21, when in a closed state and connected to the connecting member 4, has a plate thickness direction T of the portion where the pair of arms 21 are opposed and plate thickness direction T substantially coincides with the first direction B. The plate width direction X of the arms 21 where the pair of arms 21 are opposed substantially coincides with the second direction C. Since the length of the plate thickness direction T is shorter than the length of the plate width direction X, a pair of arms 21 are easily curved with respect to the plate thickness direction T. Therefore, also for this reason, the clip 2 tends to bend in the first direction B.

When the clip device 300 is inserted through the curved treatment instrument insertion channel CH of the endoscope, the clip unit 1 or the clip device 300 is rotated around the central axis O1 so that the plane AB is horizontal to the bending direction of the treatment instrument insertion channel CH. As a result, as shown in FIG. 6, the plane AB becomes horizontal to the curvature direction of the treatment instrument insertion channel CH.

As described above, both the clip 2 and the connecting member 4 tend to bend in the first direction B. As a result, by matching the curvature direction of the treatment instrument insertion channel CH and the first direction B (or plane AB), the clip unit 1 is easily passed through the treatment instrument insertion channel CH.

Next, the operation of the clip unit 1 will be described. The connecting portion 22 of the clip 2 is inserted into the inner space of the pressing tube 3 from the distal end opening 3a and is connected to the connecting member 4. With connecting portion 22 engaged by connecting member 4, the clip is pulled to the base end side of the pressing tube 3 and the pair of arms 21 is gradually closed as the pair of arms 21 are drawn into the pressing tube 3. With the pair of arms 21 positioned in the pressing tube 3 and with a portion of the length of the arms 21 remaining outside the pressing tube 3, when the traction force of the connecting portion 22 is released the self-expanding force of the pair of arms 21 acts as a restoring force and the clip 2 moves to the distal end side of the pressing tube 3 and returns to the open state.

When connecting portion 22 is further pulled to the proximal end side of the pressing tube 3, the engaging portion 24 is retracted toward the proximal end side of the pressing tube 3 and, if pulled a sufficient distance, passes the proximal end opening 3b. Since the connecting portion 22 side of the engaging portion 24 is formed in an obtuse slope, the engaging portion 24 is easily retracted toward the proximal end side and past the proximal end opening 3b. On the other hand, since the side of the engaging portion 24 oriented toward the tissue grasping portion 23 of the clip 2 is formed in a sharp slope, when the engaging portion 24 is retracted past the proximal end opening 3b, the engaging portion 24 and the proximal end opening 3b are engaged. As a result, the engaging portion 24 cannot enter the inner space of the pressing tube 3 and the pair of arms 21 is locked in a closed state. When the pair of arms 21 is locked in the closed state, the pair of arms 21 cannot return to the open state.

Cartridge

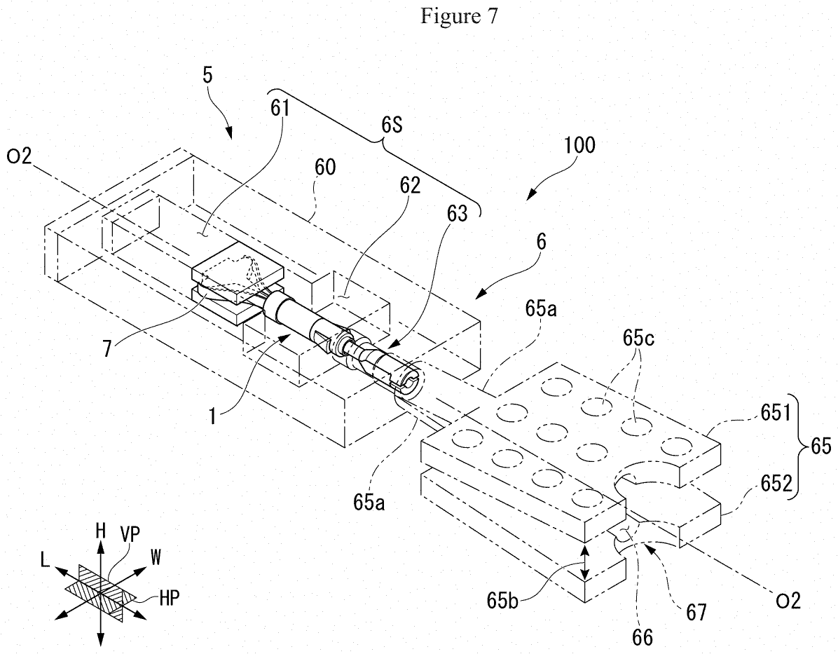

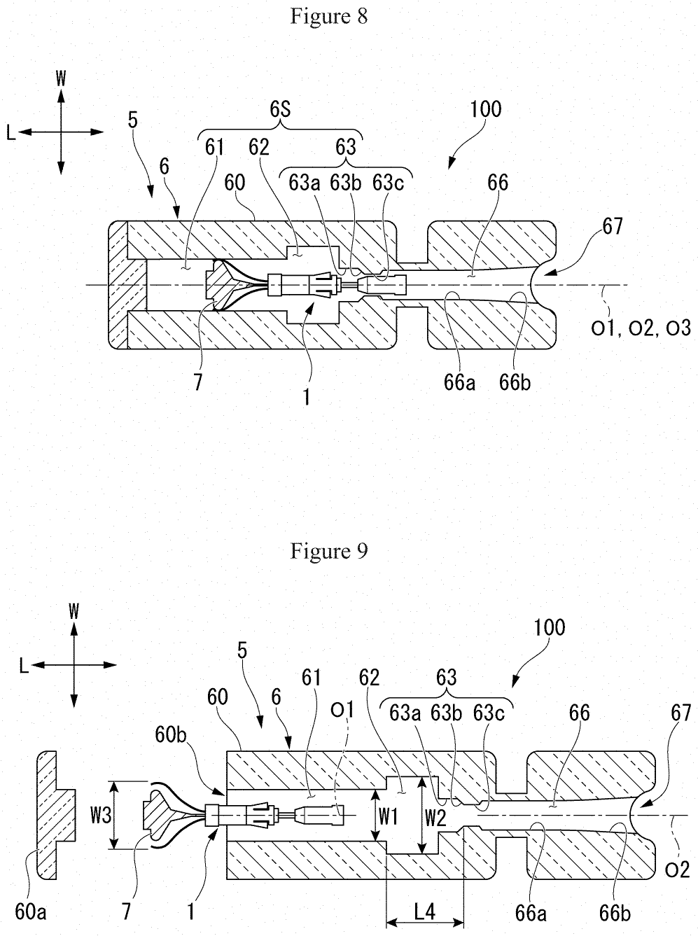

FIG. 7 is a perspective view of the cartridge 5 in which the clip unit 1 is stored. FIG. 8 is a cross-sectional view of the cartridge 5 in which the clip unit 1 is stored. The cartridge 5 includes a case 6 and a regulating member 7. In exemplary embodiments, the width of the cartridge 5 is about 10 mm to 20 mm, the length of the cartridge 5 is about 50 mm, and the thickness of the cartridge 5 is about 5 mm, as such dimensions allows the cartridge to be easily held by a hand.

As shown in FIG. 7, one of the two directions perpendicular to the longitudinal direction L of the cartridge 5 and perpendicular to each other is defined as a “width direction W′ and the other is defined as a “height direction H”. A plane horizontal to the longitudinal direction L and the width direction W is referred to as a “horizontal plane HP”. The horizontal plane in the longitudinal direction L and the height direction H is the “vertical plane VP”. Further, in the cartridge 5 in which the clip unit 1 is stored, the pair of arms 21 side is the distal end side of the cartridge 5 and the connecting member 4 side is the proximal end side of the cartridge 5.

The case 6 includes a case body 60, a compression portion 65, and a sheath connection portion 66. The case 6 is manufactured by injection molding, for example, an ABS, PC, PP, PS, an acrylic, a cycloolefin polymer, or the like, which has a moderate hardness and is made of a transparent resin material. The case 6 is formed using a transparent resin material, and the user can easily determine whether or not the clip unit 1 is present inside.

In example embodiments, case body 60 is formed in a rectangular box shape. Length in the width direction W of the case body 60 is longer than the length of the height direction H of the case body 60.

Case body 60 includes a storage area 6S in which the clip unit 1 is stored and is movable in the longitudinal direction L. The storage area 6S includes a first area 61, a second area 62, and a convolution portion 63. As shown in FIG. 8, the first area 61, the second area 62, and the convolution portion 63 are arranged from the distal end toward the proximal end in the longitudinal direction L of the case 6. The first area 61, the second area 62 and the convolution portion 63 are an internal space formed symmetrically with respect to the vertical plane VP including the central axis O2 in the longitudinal direction L of the storage region 6S.

FIG. 9 is a cross-sectional view of the cartridge 5 with the lid 60a removed. The case main body 60 has a lid 60a at its distal end. Lid 60a opens and closes the opening 60b of the distal end side in the storage area 6S of the case body 60. The clip unit 1 is stored in the storage area 6S of the case main body 60 by inserting the clip unit 1 through the opening 60b. As shown in FIG. 9, the clip unit 1 is stored in the storage area 6S with the center axis O1 of the clip unit 1 along the longitudinal direction L of the case 6. The clip unit 1 is stored in the storage area 6S with the opening/closing direction P of the pair of arms 21 coinciding with the width direction W of the case 6.

The first area 61 is an internal space in which the clip unit 1 is movably stored in the longitudinal direction L. The first area 61 communicates with the second area 62. The second area 62 is an internal space in which the clip unit 1 is movably stored in the longitudinal direction L. The length of the second area 62 in the longitudinal direction L is shorter than the length of the first area 61 in the longitudinal direction L. The second area 62 communicates with the convolution portion 63.

As shown in FIG. 9, the length W1 of the width direction W of the first area 61 is smaller than the opening width W3 of the open state of the pair of arms 21. The length W2 of the width direction W of the second area 62 is larger than the opening width W3 of the open state of the pair of arms 21. The convolution portion 63 has an enlarged diameter portion 63a, a tapered portion 63b, and a reduced diameter portion 63c. The enlarged diameter portion 63a, the tapered portion 63b, and the reduced diameter portion 63c are arranged from the distal end toward the proximal end. The enlarged diameter portion 63a is a region that allows the proximal end connecting portion 44 of the connecting member 4 to elastically spread (e.g., open and close). For example, when the connecting member 4 of the clip unit 1 and the arrowhead hook portion 231 of the clip applicator 200 is engaged, the dimensions of the enlarged diameter portion 63a allow the proximal end connecting portion 44 of the connecting member 4 to be opened and closed in a direction perpendicular to the central axis O1.

Tapered portion 63b is provided on the proximal end side of the enlarged diameter portion 63a and is formed in a tapered shape. Tapered portion 63b is enlarged toward the distal end side from the proximal end side. Therefore, when the pressing tube 3 is slid toward the proximal end side from the distal end side, the retractable wings 31 of the pressing tube 3 are housed inside the pressing tube body 30.

The reduced diameter portion 63c is a region for holding the retractable wings 31 in a retracted state. The reduced diameter portion 63c can hold the proximal end connecting portion 44 in a state in which the proximal end connecting portion 44 of the connecting member 4 is prevented from spreading when the clip unit 1 is housed in the cartridge 5.

The reduced diameter portion 63c is smoothly connected to the tapered portion 63b. When the pressing tube 3 is slid toward the reduced diameter portion 63c from the distal end side to the proximal end side, the retractable wings 31 of the pressing tube 3 contact the tapered portion 63b and become housed inside the pressing tube body 30. Therefore, it is possible to transition the retractable wing 31 of the pressing tube 3 to a housing state.

As shown in FIG. 7, compression portion 65 is a plate-like member provided on the proximal end of the case body 60. The compression portion 65 has a first compression section 651 and a second compression section 652. The first compression portion 651 and the second compression portion 652 are provided so as to face each other in the height direction H of the case 6.

The compression portion 65 has a connecting portion 65a connected to the case body 60. The connecting portion 65a connects the first compression section 651 and the second compression section 652 to the case body 60 separately. The connecting portion 65a is bent such that the first compression section 651 and the second compression section 652 are separated from each other. Therefore, a space 65b is formed between the first compression section 651 and the second compression section 652. The first compression section 651 and the second compression section 652 are more separated from each other on the proximal end side than on the distal end side.

The first compression section 651 and the second compression section 652 are, for example, about 20 mm square and are formed to have a size suitable for being pinched by a finger. The outer surface of the first compression section 651 and the second compression section 652 includes, for example, a plurality of concave features 65c, such as hemispherical depressions, and provide a non-slip surface during squeezing.

The sheath connection portion 66 is an insertion groove into which the sheath 220 can be inserted. Sheath connection portion 66 is an arcuate groove formed on the inner surface of the first compression section 651 and the second compression section 652, and communicates with the reduced diameter portion 63c of the convolution portion 63 in the storage area 6S. Sheath connection portion 66 has a straight portion 66a having an unchanged diameter, and a tapered portion 66b which gradually increases in diameter toward the entrance 67 of the proximal end side. The sheath 220 enters the straight portion 66a via the tapered portion 66b and abuts against the proximal end side of the reduced diameter portion 63c. The inner diameter of the entrance 67 is, for example, 3 mm or more.

The user can fix the sheath 220 to the case 6 by squeezing the first compression section 651 and the second compression section 652 in a state in which the sheath 220 is inserted into the sheath connecting portion 66 from the entrance 67.

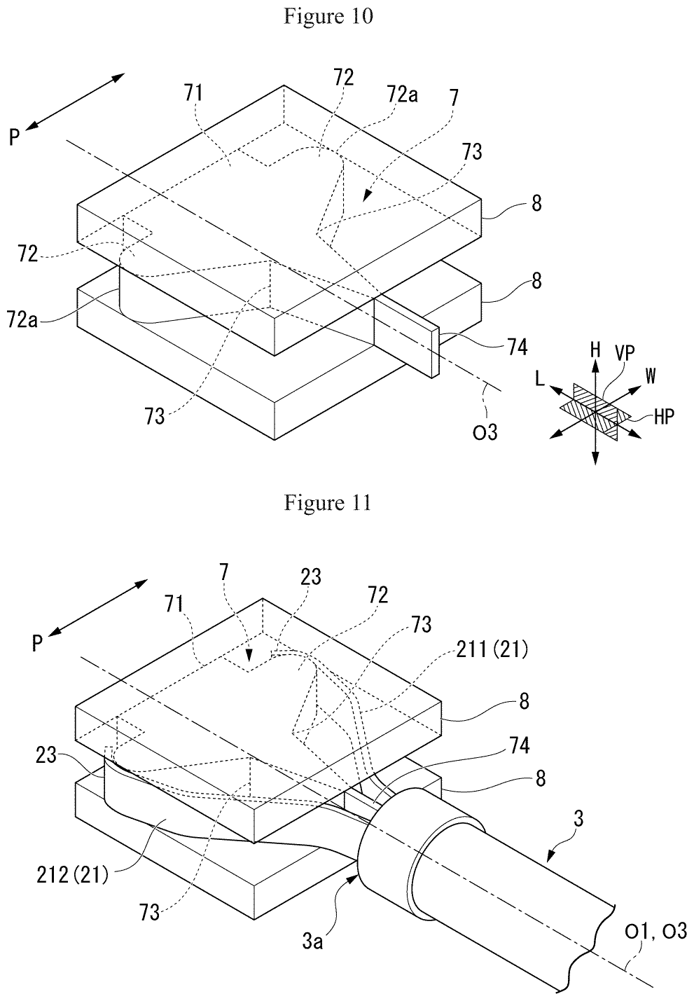

FIG. 10 is a perspective view of the regulating member 7. The regulating member 7 is movably stored in the first area 61 and the second area 62 together with the clip unit 1. The regulating member 7 is formed of, for example, a resin similar to that of the case 6. To facilitate viewing the regulating member 7 when within case 6, the regulating member 7 may not be formed of the same transparent resin as used for case 6.

Regulating member 7, as shown in FIG. 10, is formed in a shape symmetrical with respect to the vertical plane VP including the central axis O3. Regulating member 7 includes a distal end portion 71, a protruding portion 72, a tapered portion 73, and a pressing portion 74. The distal end portion 71 and the protruding portion 72 and the tapered portion 73 and the pressing portion 74 are arranged toward the proximal end from the tip along the central axis O3 direction of the regulating member 7.

FIG. 11 is a perspective view of the regulating member 7 held by the pair of arms 21. The regulating member 7 is gripped by the clip 2 with the center axis O3 substantially coincident with the center axis O1 of the clip unit 1. As shown in FIG. 8, the clip unit 1 is stored in the storage area 6S with the clip 2 gripping the regulating member 7. In this arrangement, it is desirable that the central axis O1, the central axis O2, and the central axis O3 coincide with each other.

Distal end portion 71 is provided to protrude from the protruding portion 72 to the distal end side. The distal end of the distal end portion 71 is located at the distal end of the tissue grasping portion 23 of the clip 2 that grasps the regulating member 7. Therefore, the distal end portion 71 prevents the tissue grasping portion 23 of the clip 2 from coming into contact with the case body 60. Further, the tip of the distal end portion 71 is formed in a plane perpendicular to the central axis O3.

Protruding portion 72 is a member protruding in a direction perpendicular to the central axis O3 (hereinafter, referred to as “protruding direction P”). Protruding portions 72 are provided on both sides of the central axis O3. The protruding portion 72 is gripped by the first arm 211 and the second arm 212 of the clip 2. Opening and closing direction P of the pair of arms 21 for gripping the protruding portion 72 substantially coincides with the protruding direction P of the protruding portion 72. Curvature of the outer peripheral surface of the protruding portion 72 is smaller than the curvature of the inner peripheral surface of the tissue grasping portion 23. Therefore, the pair of arms 21 can reliably grip the protruding portion 72.

Tapered portion 73 is a member formed in a tapered shape. Tapered portions 73 are provided on both sides across the central axis O3. Tapered portion 73 has a short length in the protruding direction P with respect to the protruding portion 72. Tapered portion 73, toward the proximal end side from the distal end side, has a length in the protruding direction P that is shortened.

Pressing portion 74 is a projecting member for regulating the minimum approach distance between the clip 2 and the pressing tube 3. Pressing portion 74 is provided on the proximal end side of the tapered portion 73. When engaging the edge of the distal end opening 3a, pressing portion 74 does not enter the inner space of the pressing tube 3 from the distal end opening 3a. Therefore, even when the clip 2 is pulled in a direction approaching the pressing tube 3, the pressing portion 74 engages the edge of the distal end opening 3a, which regulates the minimum approach distance between the clip 2 and the pressing tube 3.

As shown in FIG. 10, the regulating member 7 is sandwiched by the auxiliary member 8 from the height direction H. As the regulating member 7 is gripped by a pair of arms 21, auxiliary member 8 adjusts the position of the height direction H of the regulating member 7. When it is not necessary to adjust the position of the regulating member 7 in the height direction, the auxiliary member 8 is unnecessary.

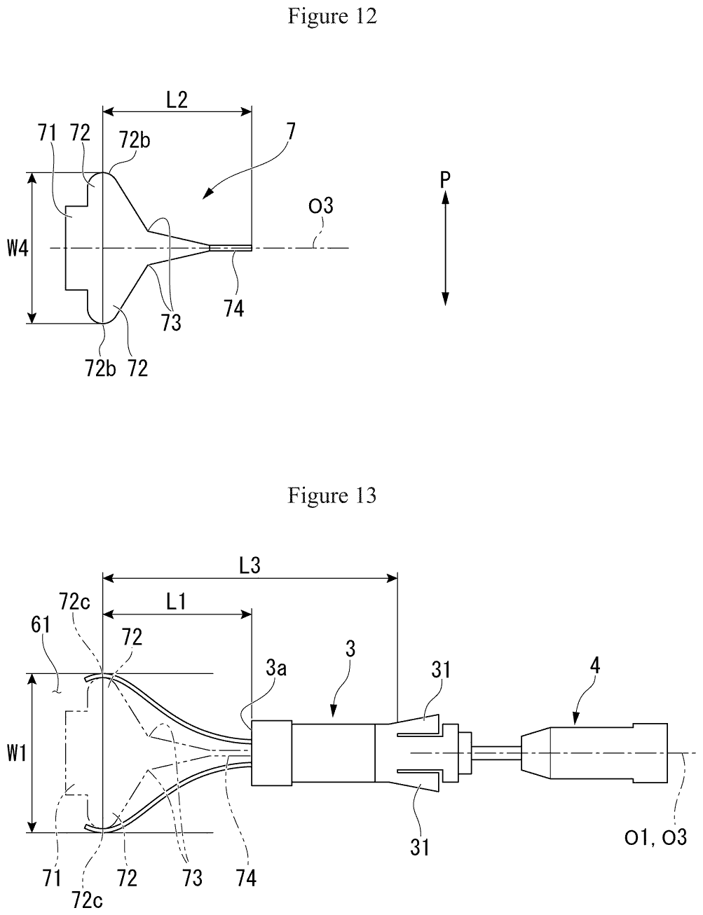

FIG. 12 is a plan view of the regulating member 7. In the protruding portion 72, the maximum protruding point 72b is the most protruding portion in the protruding direction P from the central axis O3. Length W4 between the maximum protruding point 72b is slightly smaller than the length W1 of the width direction W of the first area 61. The length in the central axis O3 direction from the maximum protruding point 72b to the base end of the pressing portion 74 is L2.

FIG. 13 is a plan view of the regulating member 7 gripped in the first area 61. The regulating member 7 is stored in the first area 61 in a state of being gripped by the pair of arms 21. Length W1 in the width direction W of the first area 61 is smaller than the opening width W3 of the open state of the pair of arms 21. Therefore, the pair of arms 21 grip the regulating member 7 in the closed state from the open state. Opening width of the pair of arms 21 is about the length W1 of the width direction W of the first area 61. A pair of arms 21 abuts the case body 60 in the opening and closing direction P.

The length in the central axis O3 direction from the contact point 72c of the pair of arms 21 and the case body 60 to the base end of the pressing portion 74 is L1. Length L1 in the central axis O3 direction from the contact point 72c to the base end of the pressing portion 74 is preferably equal to or more than the length L2 in the central axis O3 direction from the maximum protruding point 72b to the base end of the pressing portion 74 (L1≥L2). Because L1≥L2, when the clip 2 is pulled proximally, the clip 2 is securely engaged with the regulating member 7.

As shown in FIGS. 12 and 13, the length W4 between the maximum protruding point 72b is slightly smaller than the length W1 in the width direction W of the first area 61. Therefore, the regulating member 7 is maintained in a state of being gripped by the pair of arms 21 in the first area 61.

The length in the central axis O1,O3 from the contact point 72c to the distal end of the retractable wing 31 (see FIG. 13) is L3. Length L3 in the central axis O1,O3 from the contact point 72c to the distal end of the retractable wing 31 is preferably equal to or greater than the distance L4 from the distal end of the second area 62 to the proximal end of the tapered portion 63b (L3≥L4) (see FIG. 9).

Loading Method of Clip Unit

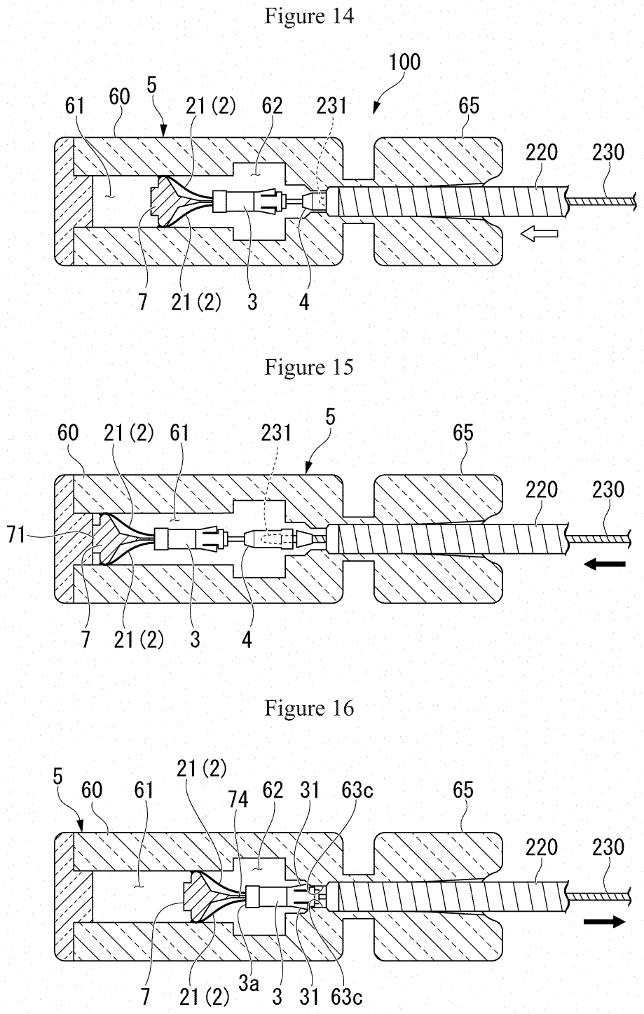

Next, the operation of the cartridge system 100 will be described. FIGS. 14 to 25 are diagrams for explaining a method of loading the clip unit 1 into the clip applicator 200 using the cartridge 5.

As shown in FIG. 14, the user inserts the sheath 220 of the clip applicator 200 into the storage area 6S of the case 6 from the sheath connection portion 66. The user squeezes the sheath 220 with the compression portion 65, and fixes the sheath 220 to the case 6.

As shown in FIG. 15, by operating the operation unit 240 to advance the operation wire 230 with respect to the sheath 220, the user advances the arrowhead hook portion 231. The arrowhead hook portion 231 is connected to the connecting member 4 of the clip unit 1.

The tip of the distal end portion 71 of the regulating member 7 is formed in a plane perpendicular to the central axis O3. Therefore, even when the tip of the clip unit 1 is pushed to the distal end side by the arrowhead hook portion 231 and is in contact with the case body 60, the central axis O1 of the clip unit 1 is hardly displaced with respect to the central axis O2 of the storage area 6S.

As shown in FIG. 16, the user pulls the operation wire 230. The clip 2 of the clip unit 1 is pulled proximally by the connecting member 4 connected to the arrowhead hook portion 231. Hook 41f of the connecting member 4 tows the connecting portion 22 of the clip 2 without breaking. The regulating member 7 moves in the first area 61 of the storage region 6S in a state of being in contact with the pair of arms 21. Since the pair of arms 21 has a self-expanding force and is engaged with the edge of the distal end opening 3a of the pressing tube 3, the pressing tube 3 together with the clip 2 is also towed to the proximal end side.

In the first area 61, pair of arms 21 abuts the case body 60 in the opening and closing direction P. Contact between the pair of arms 21 and the case body 60 generates a frictional force. This frictional force prevents the clip 2 from being pulled into the internal space of the pressing tube 3. In other words, the clip 2 can be prevented from being locked in the closed state by the pressing tube 3.

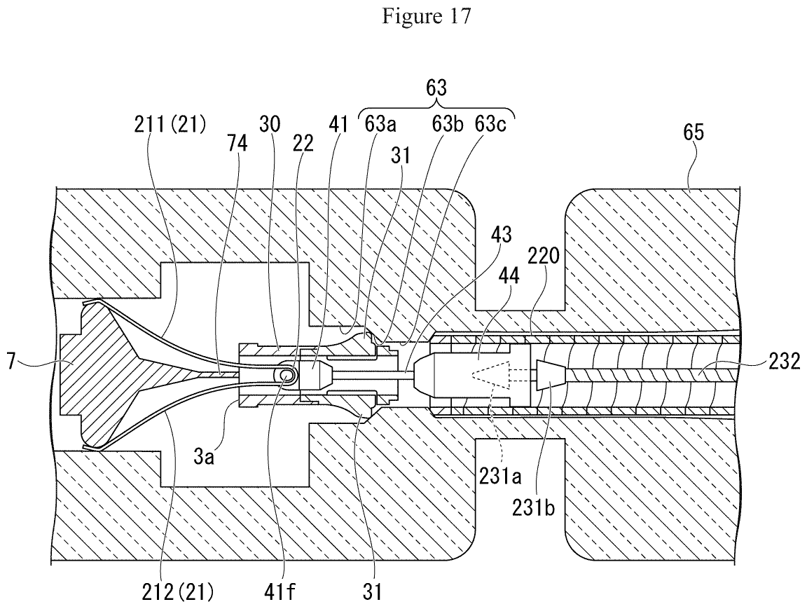

As shown in FIG. 17, when the clip 2 is towed to the proximal end side, since the pressing portion 74 of the regulating member 7 is engaged with the edge of the distal end opening 3a of the pressing tube 3, the minimum approach distance between the clip 2 and the pressing tube 3 is restricted. Pressing portion 74 is in contact with the pressing tube 3 in a state of being in contact with the pair of arms 21 by traction of the operation wire 230, to regulate the relative movement of the regulating member 7 with respect to the pressing tube 3. This also advantageously prevents the clip 2 pulled proximally by the connecting member 4 from being pulled into the inner space of the pressing tube 3 and from being locked in the closed state by the pressing tube 3.

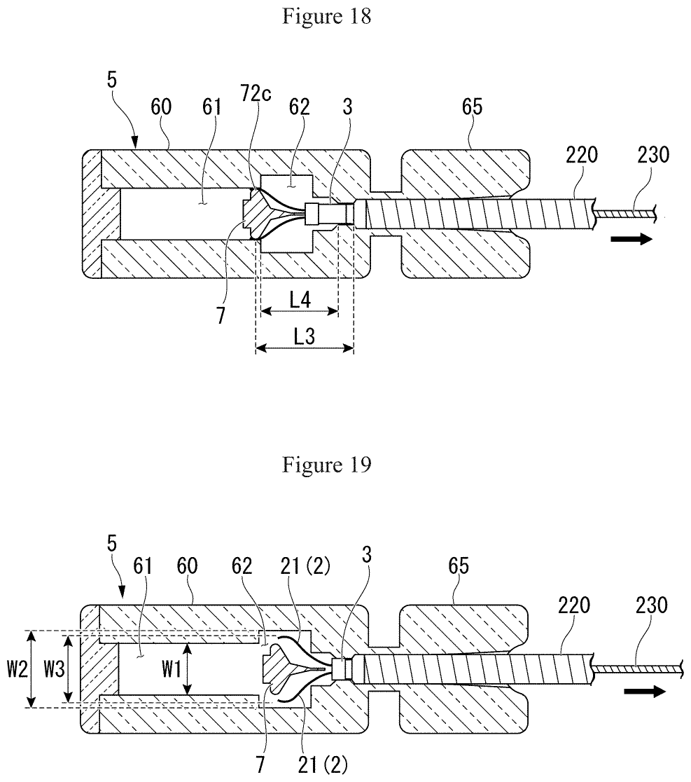

The user further pulls the clip unit 1 proximally. As shown in FIG. 18, the pressing tube 3 passes through the convolution portion 63. As pressing tube 3 is slid toward the reduced diameter portion 63c from the distal end side to the proximal end side, the retractable wings 31 are housed inside the pressing tube body 30. The pressing tube 3, with the retractable wings 31 now housed inside the pressing tube body 30, is pulled into the sheath 220.

As shown in FIG. 18, in the central axis O1,O3 direction, length L3 from the contact point 72c to the distal end of the retractable wing 31 is equal to or greater than length L4 from the distal end of the second area 62 to the proximal end of the tapered portion 63b. Therefore, when the retractable wings 31 are housed inside the pressing tube body 30, the contact point 72c between the pair of arms 21 and the case body 60 is located in the first area 61. That is, until the retractable wing 31 is housed inside the pressing pipe body 30 and a pair of arms 21 grips the regulating member 7, it is not drawn into the inner space of the pressing tube 3.

As shown in FIG. 19, the user further pulls the operation wire 230 to pull the regulating member 7 to the second area 62. Length W2 in the width direction W of the second area 62 is larger than the opening width W3 of the open state of the pair of arms 21. Therefore, the regulating member 7 is not gripped by the pair of arms 21 in the second area 62.

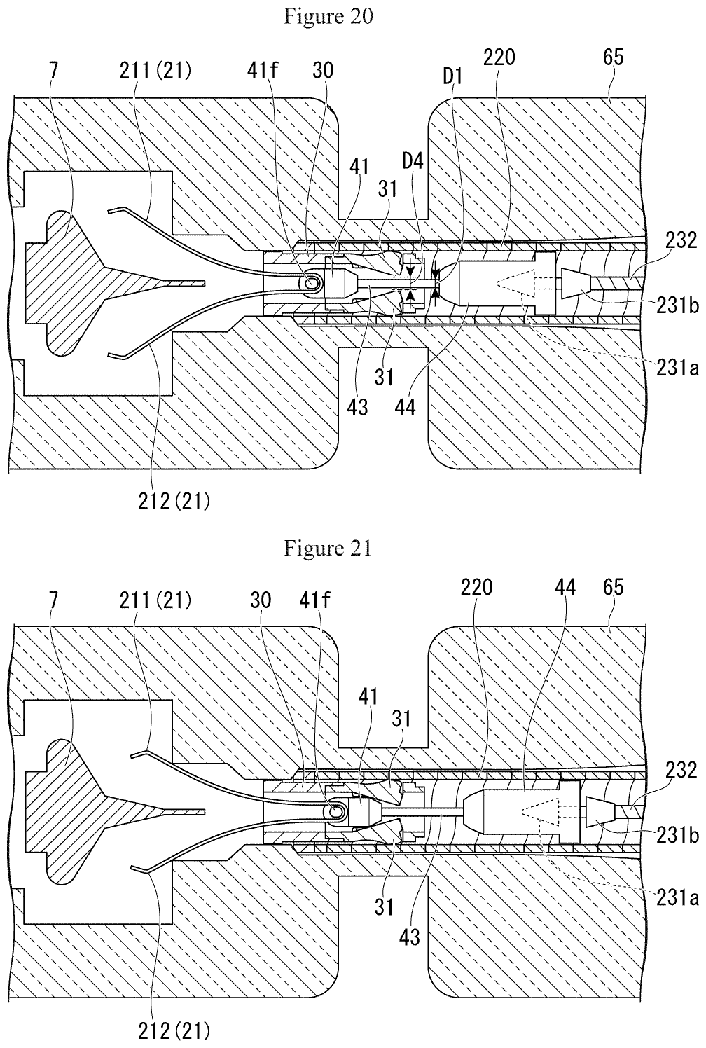

As shown in FIG. 20, the retractable wings 31 are pressed by the inner peripheral surface of the sheath 220 to be in a retracted state. The first length D1 of the rod-shaped portion 43 in the first direction B is shorter than the fourth length D4 between the two retractable wings 31 in the retracted state. Since the retractable wings 31 can be sufficiently recessed radially inward, friction generated between the retractable wings 31 and the inner peripheral surface 220b of the sheath 220 is reduced, and the user can easily move the clip unit 1 along the inner peripheral surface 220b of the sheath 220.

If the retractable wings 31 arranged around the central axis O1 do not coincide with the first direction B of the rod-shaped portion 43, the retracting of the retractable wings 31 contacts the rod-shaped portion 43 and the pressing tube 3 is rotated about the central axis O1. As a result, the retractable wing 31 is disposed in the first direction B of the rod-shaped portion 43.

The user further pulls the clip unit 1 proximally. As shown in FIG. 21, the retractable wing 31 is in the retracted state and is engaged with the distal end connecting portion 41. Distal end connecting portion 41 can be reliably towed to the proximal end side A2 of pressing tube 3 when engaged with the retractable wing 31 in its retracted state.

FIG. 22 is an enlarged view of the pressing tube 3 and the like shown in FIG. 21. The fourth length D4 in the first direction B between the two retractable wings 31 in the retracted state is shorter than the third length D3 of the first direction B of the distal end connecting portion 41 and is longer than the first length D1 of the first direction B of the rod-shaped portion 43 (D3>D4>D1). In the retracted state, retractable wing 31 contacts the cylindrical portion 41c of the distal end connecting portion 41 and does not contact the rod-shaped portion 43. Since the contact area between the connecting member 4 and the retractable wing 31 is small, when the connecting member 4 is towed, the force for connecting member 4 to push the retractable wing 31 radially outward is reduced.

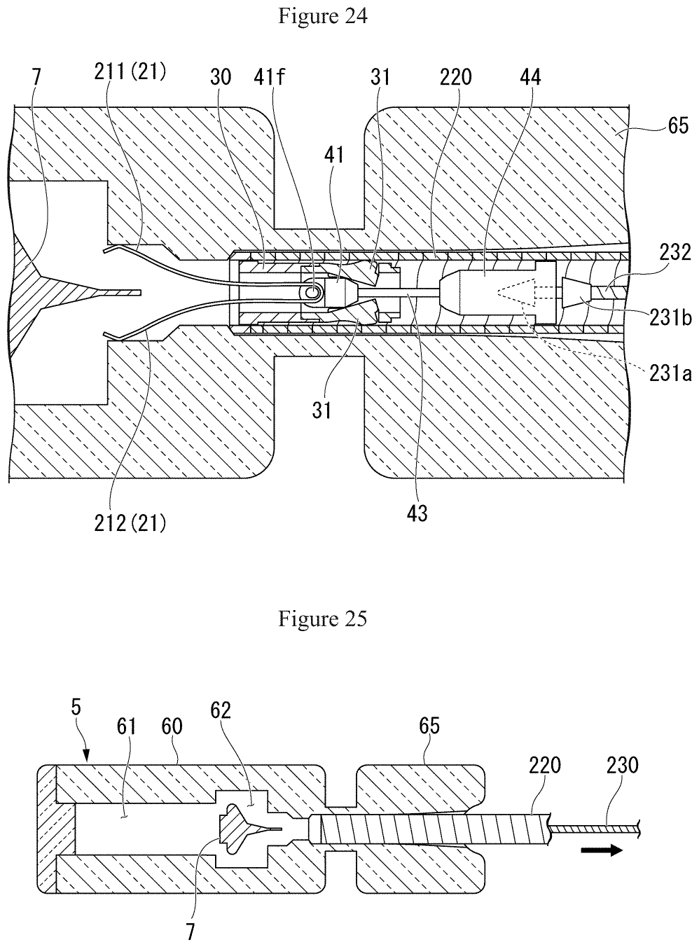

The user further tows the operating wire 230 as shown in FIGS. 23 and 24. The clip 2 is pulled proximally separately from the regulating member 7. The tapered portion 73 of regulating member 7 is formed on the proximal end side of the protruding portion 72 which has been gripped by the pair of arms 21. Therefore, when the regulating member 7 is pulled to the proximal end side, the pair of arms 21 are unlikely to be caught by the regulating member 7.

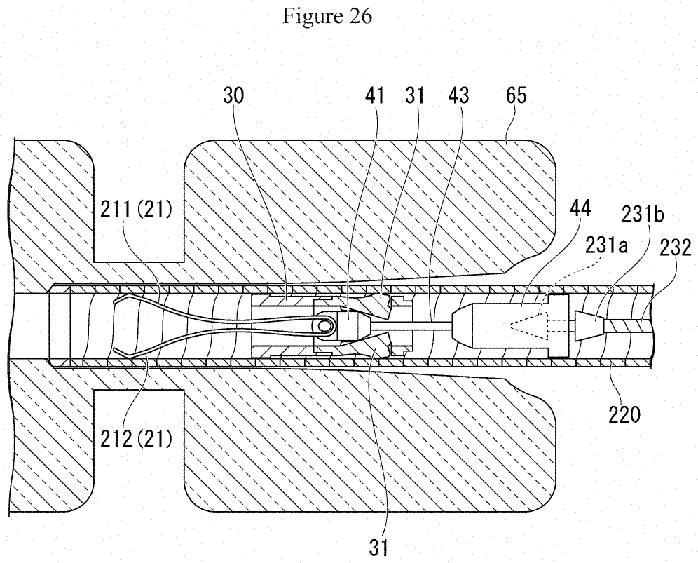

As shown in FIG. 25, the user further pulls the operation wire 230. Since the pair of arms 21 does not grip the regulating member 7, the minimum approach distance between the clip 2 and the pressing tube 3 is not regulated. As shown in FIG. 26, the clip 2 pulled proximally is pulled into the sheath 220 while being pulled into the inner space of the pressing tube 3. The clip 2 may be pulled into the sheath 220 without being pulled into the interior space of the pressing tube 3. This completes the loading of the clip unit 1 into the clip applicator 200. The user releases the squeezing of the sheath 220 by the compression portion 65, and pulls the sheath 220 out from the case 6.

Operation and Operation of Clip Unit

Next, the operation and operation of the clip unit 1 will be described with reference to FIGS. 27 to 31.

As shown in FIG. 26, the connecting member 4 of the loaded clip unit 1 is connected to the arrowhead hook portion 231 inserted through the sheath 220. The retractable wings 31 are pressed by the inner peripheral surface of the sheath 220 to be in a retracted state.

The pair of arms 21 of the loaded clip unit 1 is in a closed state by being pressed by the inner peripheral surface of the sheath 220. The engaging portion 24 is located on the distal end side rather than the proximal end opening 3b, and the pair of arms 21 is not locked in a closed state.

FIG. 27 shows the clip unit 1 in a states as would be after introduction into a body. The user introduces the clip unit 1 loaded in the sheath 220 into the body via the treatment instrument insertion channel of the endoscope. Next, the user advances the arrowhead hook portion 231 by advancing the slider 242 along the operation unit main body 241. The user advances the clip unit 1 until the retractable wings 31 exit the sheath 220. By exiting the sheath 220, the retracted retractable wings 31 return to the projecting state, which is a basic posture from the immersion state. As the distal end of the pair of arms 21 comes out of the sheath 220, the self-expanding force of the pair of arms 21 acts as a restoring force and the clip 2 returns to the open state while moving further to the distal end side with respect to the pressing tube 3. Even when the pair of arms 21 maximally protrudes from the pressing tube 3 in the open state, the engaging portion 24 is disposed in the inner region S of the pressing tube 3.

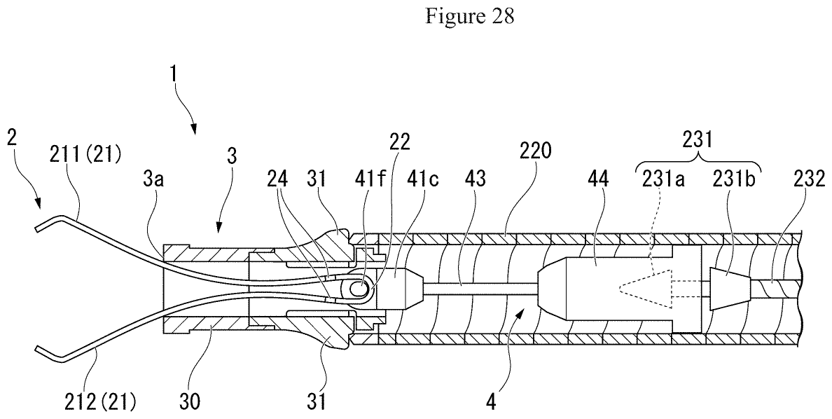

FIG. 28 is a view showing the clip unit 1 in which the pair of arms 21 are closed. The user retracts the arrowhead hook portion 231 by retracting the slider 242 along the operation unit main body 241. The connecting member 4 connected to the arrowhead hook portion 231 pulls the clip 2. The pair of arms 21 have a self-expanding force and, by being towed to the proximal end side, pushes the distal end opening 3a of the pressing tube 3 to the proximal end side. The retractable wings 31 in the projecting state engage with the sheath 220, so that the retractable wings 31 are not pulled into the sheath 220. Therefore, the clip 2 pulled by the connecting member 4 is pulled into the pressing tube 3.

Connecting portion 22 of the clip 2 is hooked by the connecting member 4 and the clip 2 is towed to the base end side of the pressing tube 3, drawing pair of arms 21 into the pressing tube 3 such that the pair of arms 21 is gradually closed. From this state, when the traction force of the connecting portion 22 is released, the self-expanding force of the pair of arms 21 acts as a restoring force and the clip 2 returns to the open state while moving to the distal end side. In this way, the user can return the pair of arms 21 to the open state to re-grasp the tissue.

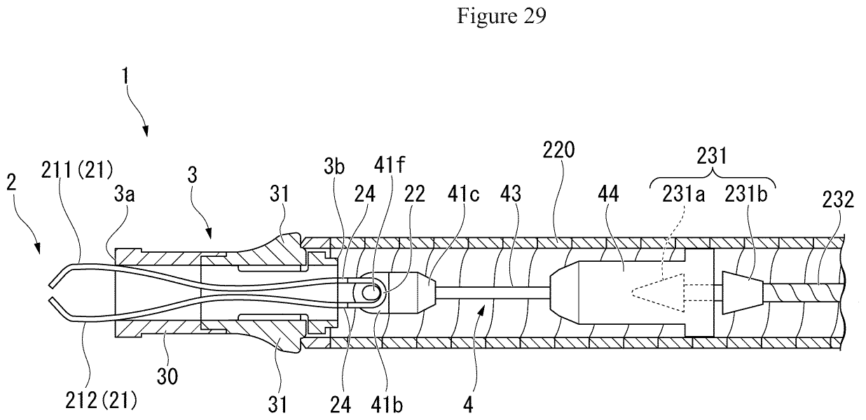

FIG. 29 is a diagram showing the clip unit 1 in which the clip 2 is locked. By further towing connecting portion 22 to the proximal end side of the pressing tube 3, the engaging portion 24 is drawn to the proximal end side from the proximal end opening 3b. Since the connecting portion 22 side of the engaging portion 24 is formed with an obtuse slope, the engaging portion 24 is easily retracted to the proximal end side from the proximal end opening 3b. On the other hand, since the tissue grasping portion 23 side of the engaging portion 24 is formed with a sharp slope, when the engaging portion 24 is retracted to the proximal end side from the proximal end opening 3b, the engaging portion 24 and the proximal end opening 3b is engaged. As a result, the movement of the clip 2 toward the distal end side with respect to the pressing tube 3 is restricted, and the pair of arms 21 are locked in the closed state. When the pair of arms 21 is locked in the closed state, the pair of arms 21 cannot return to the open state.

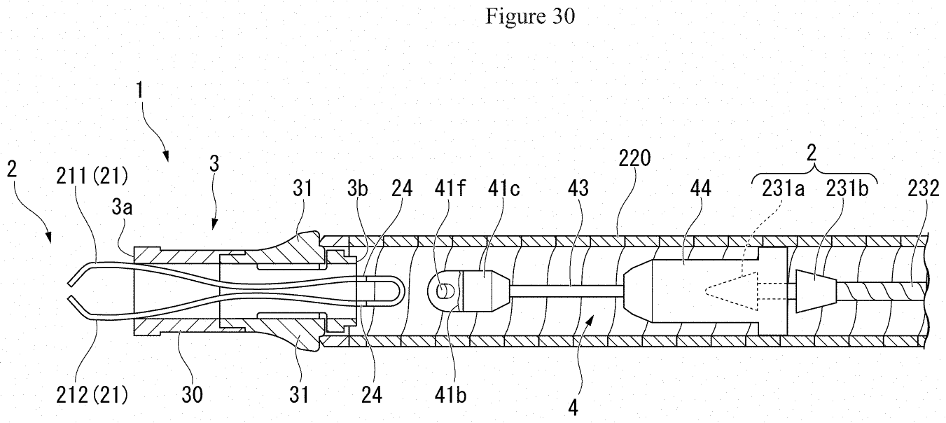

FIG. 30 is a diagram showing the clip unit 1 in which the clip 2 is separated from the connecting member 4. The user further pulls on the clip 2. When the amount of breaking force, i.e., tensile force, applied to the proximal end side of the breakage portion 41b by pulling connecting portion 22 relative to the hook 41f exceeds a predetermined amount, such as up to 90N (Newton), for example 20N. the breaking portion 41b is broken. The breaking strength of the breaking portion 41b is lower than the breaking strength of the rod-shaped portion 43. Therefore, the breaking portion 41b is broken, rather than the rod-shaped portion 43.

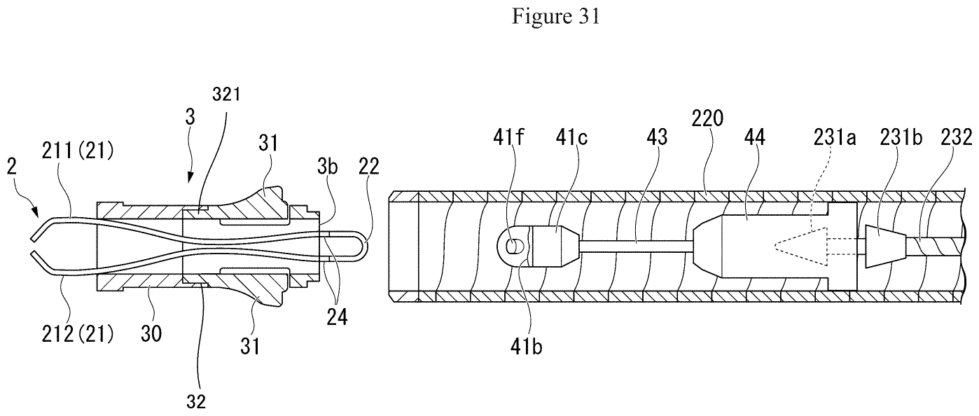

FIG. 31 is a view showing the clip unit 1 after breaking. The user retracts the sheath 220 and places the clip 2, which is in a ligated state of the tissue, in the body.

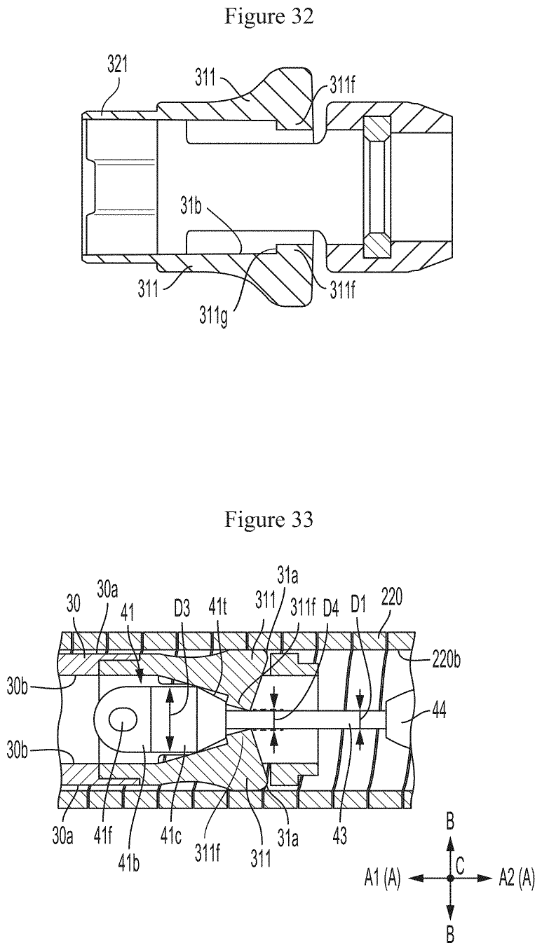

FIG. 32 is a sectional view of an alternative embodiment of a retractable wing. The alternative embodiment of retractable wing 311 is similar to retractable wing 31 shown and described with respect to FIG. 22, but the alternative embodiment of retractable wing 311 includes a projection portion 311f. The projecting portion 311f is a portion of the retractable wing 311 that extends radially inward (relative to the central axis O1) in the first direction B from a proximal end of the inner peripheral surface 31b of the retractable wing 311. At a distal end of the projecting portion 311f, the projecting portion 311f has a step surface 311g. The step surface 311g forms an angle with the inner peripheral surface 31b that is equal to or less than 115 degrees, alternatively equal to or less than 90 degrees.

As seen in FIG. 33, when the retractable wing 311 is in a retracted state, the body of the retractable wings 311 are located within the envelope of the pressing tube body 30. In the retracted state, the projecting portion 311f can engage the connecting member 4. For example, the step surface 311g can engage a portion of the tapered surface 41t, such as a proximal end surface 41t1 of the tapered surface 41t. Because of the engagement between projecting portion 311f and connecting member 4, when the connecting member 4 is pulled toward the proximal end side, the pressing tube 3 also moves toward the proximal end side. Further, the engagement between step surface 311g and proximal end surface 41t1 reliably transmits the translational force from the connecting member 4 to the pressing tube 3.

According to the cartridge system 100 of the present embodiment, the first length D1 in the first direction B of the rod-shaped portion 43 is shorter than the second length D2 in the second direction C, and is more likely to bend in the first direction B than in the second direction C (D2>D1). By matching the curvature direction of the treatment instrument insertion channel with the first direction B, the clip unit 1 is easily passed through the treatment instrument insertion channel that is curved.

According to the cartridge system 100 of the present embodiment, the first length D1 of the first direction B of the rod-shaped portion 43 is shorter than the fourth length D4 between the two retractable wings 31,311 in the retracted state (D4>D1). Since the retractable wings 31,311 can be immersed sufficiently radially inwardly, the user can easily move the clip unit 1 along the inner circumferential surface of the sheath 220.

According to the cartridge system 100 of the present embodiment, the fourth length D4 between the two retractable wings 31,311 that are in the retracted state is shorter than the third length D3 of the first direction B of the distal end connecting portion 41 and is longer than the first length D1 of the first direction B of the rod-shaped portion 43 (D3>D4>D1). Thus, retractable wings 31,311 in the retracted state can be towed to the proximal end side A2 and pressing tube 3 is reliably engaged with the distal end connecting portion 41.

Construction and connection of parts of the pressing tube.

Next, connection of parts of the pressing tube 3 will be described with reference to FIGS. 31 to 40A-G. In some embodiments, the pressing tube 3 is formed of a unitary body including a section with the pressing tube body 30 and the section with the retractable wings 31. In alternative embodiments, the pressing tube 3 is formed by assembling a plurality of parts to form the pressing tube 3. For example and as shown in FIG. 31, the pressing tube 3 includes a section with the pressing tube body 30 and a section with the retractable wings 31 and the two sections are joined at an interface 32. Here, the pressing tube 3 is assembled by inserting a male part 321 of the section with the retractable wings 31 into an opening in the section with the pressing tube body 30. The opening in the section with the pressing tube body 30 functions as a female part to receive the male part 321 of the section with the retractable wings 31. The outer diameter surface of the male part 321 is in press-fit contact with the inner diameter surface of the opening in the section with the pressing tube body 30. Such a press-fit contact provides a secure connection between the two parts.

In some embodiments, the entire outer diameter surface of the male part 321 is at the same radial distance, i.e., a first radial distance, from the axial centerline of the pressing tube 3 and the entire inner diameter surface of the opening in the section with the pressing tube body 30 is at the same radial distance, i.e., a second radial distance, from the axial centerline of the pressing tube 3 and, when fitted together, a press-fit contact is formed between the outer diameter surface of the male part 321 and the inner diameter surface of the opening in the section with the pressing tube body 30 that extends uninterrupted about the interface 32. However, when the amount of surface area in the press-fit contact is high, a large amount of friction can be encountered during assembly, making it difficult to assemble the pressing tube 3. Additionally, if the amount of surface area in the press-fit contact is high, a rigidity of the assembled pressing tube 3 extends along the length of both parts and, when a clip device 300 with such an assembled pressing tube 3 is being passed through treatment instrument insertion channel CH of the curved endoscope (see FIG. 6), this rigid length can result in stresses forming that can break the pressing tube 3.

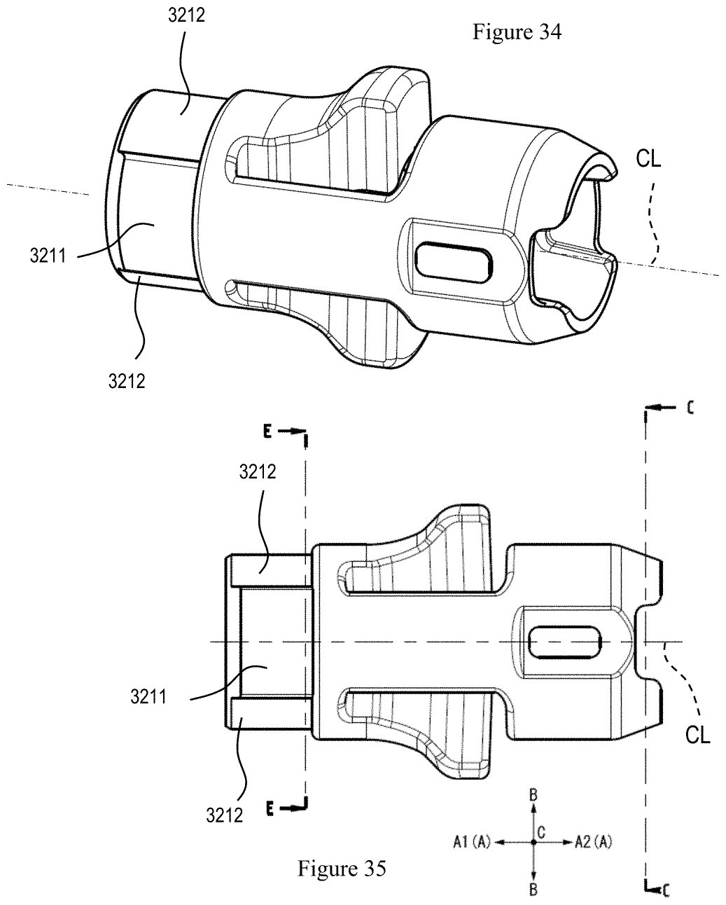

In alternative embodiments, the amount of surface area in press-fit contact is reduced by extending the press-fit contact about less than the entire circumference of the interface 32. FIG. 34 is a perspective view of a section with the retractable wings 31 and FIG. 35 is a side view of a section with the retractable wings 31. As shown in FIGS. 34 and 35, the outer diameter surface of the male part 321 can have regions that are at different radial distances from the axial centerline CL of the pressing tube 3. For example, a first region 3211 is at a first radial distance and a second region 3212 is at a second radial distance, and the first radial distance is greater than the second radial distance. Typically, there are more than one first regions 3211 circumferentially separated from each other by a second region 3212. When assembled with the pressing tube body 30, only the outer diameter surfaces of the male part 321 in the first regions 3211 are in press-fit contact with the inner diameter surface of the opening in the section with the pressing tube body 30, and the outer diameter surfaces of the male part 321 in the second regions 3212 are not in contact with the inner diameter surface of the opening in the section with the pressing tube body 30. Thus, the overall surface area that is part of the press-fit contact is reduced.

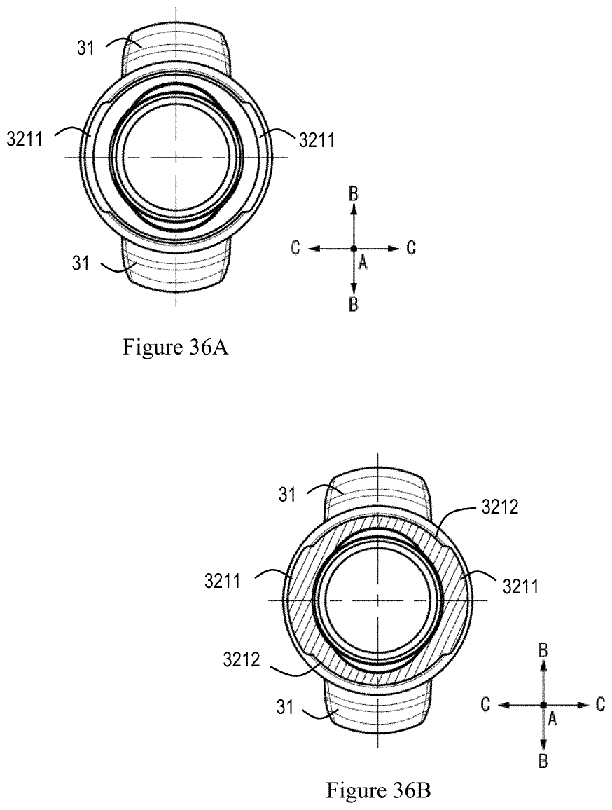

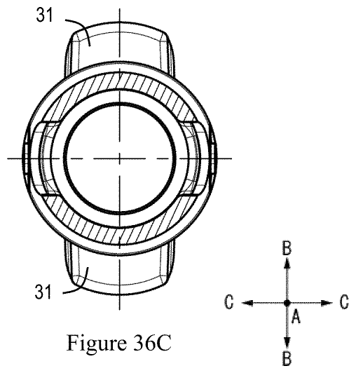

FIG. 36A is an end view of the male part 321 seen along axial centerline CL in the A2 direction, FIG. 36B is a cross-sectional view of the male part 321 taken at E-E in FIG. 35 and seen along axial centerline CL in the A2 direction, and FIG. 36C is a cross-sectional view of the male part 321 taken at C-C in FIG. 35 and seen along axial centerline CL in the A1 direction. In both FIGS. 36A and 36B, the first regions 3211 and second regions 3212 are visible. The circumferential position of the first regions 3211 are different from the circumferential position of the retractable wings 31. In some embodiments, the circumferential center of the first regions 3211 are 90 degrees offset from the circumferential center of the retractable wings 31.

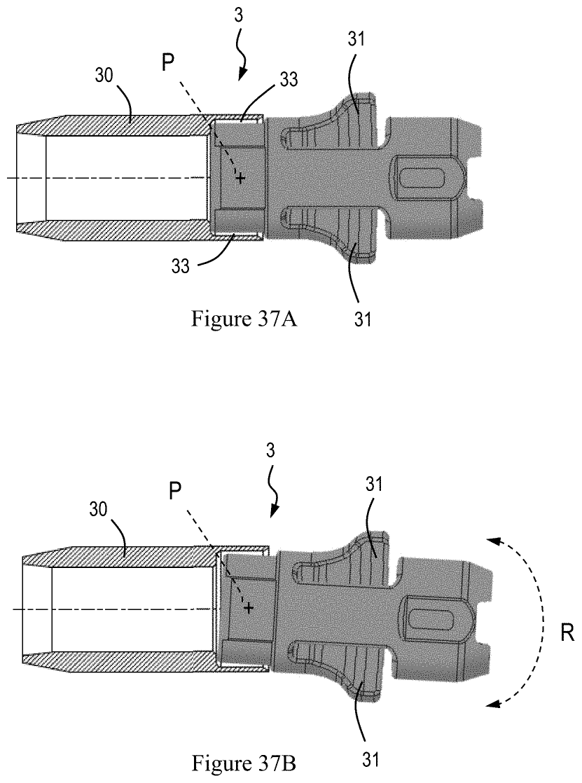

As previously noted (see FIG. 6 and related discussion), the clip unit 1 tends to bend in the direction B or in the plane AB. In the embodiment with first regions 3211 and second regions 3212, the press-fit contact by first regions 3211 functions as a pivot point P about which a first part of the pressing tube 3 can rotate R relative to the second part of the pressing tube 3, e.g., the section with the retractable wings 31 can pivot relative to the section with the pressing tube body 30. FIGS. 37A and 37B show these sections in alignment (FIG. 37A) and pivoted (FIG. 37B). When pivoting, the circumferential position of the second regions 3212 provide a space 33 to accommodate the pivoting motion of the section with the retractable wings 31.

Recall the rod-shaped portion 43, in a cross-section perpendicular to the longitudinal direction A, has a surface with a first length D1 that is a shorter rectangular shape or elliptical shape than the second length D2 (see FIGS. 4 and 5). Here, the circumferential position of the first regions 3211 is located facing the side of the rod-shaped portion 43 that has the shorter first length. By having the first regions 3211 and the first length D1 side of the rod-shaped portion 43 aligned or substantially (±15 degrees) aligned, the non-contacting second regions 3212 and the side of the rod-shaped portion 43 that has the shorter first length are similarly aligned or substantially (±15 degrees) aligned and, as such, cooperate to improve the flexibility of the clip device 300 in the assembled pressing tube 3 when being passed through treatment instrument insertion channel CH of the curved endoscope (see FIG. 6).

In alternative embodiments, the opening is in the section with the retractable wings 31 and the male part is on the section with the pressing tube body 30, and the pressing tube 3 is assembled by inserting the male part 321 of the section with the pressing tube body 30 into the opening in the section with the retractable wings 31.

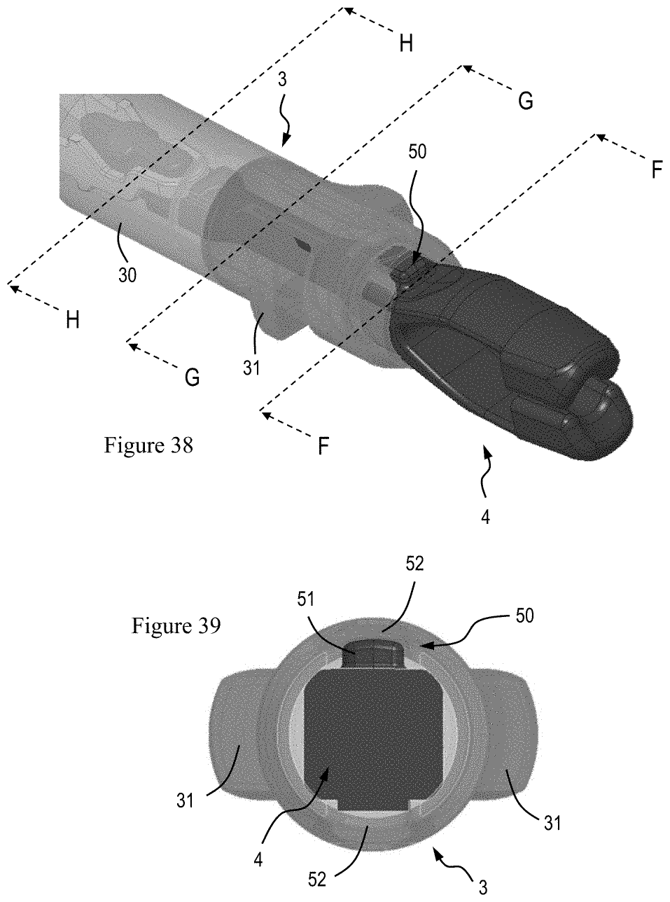

To ensure the above alignment or substantial alignment of the first regions 3211 and the second regions 3212 with the respective corresponding surfaces of the rod-shaped portion 43, an alignment structure can be incorporated. The alignment structure facilitates proper alignment of the first regions 3211 and the second regions 3212 with the respective corresponding surfaces of the rod-shaped portion 43 during assembly as well as restricts relative rotation of the connecting member 4 and pressing tube 3. For example, an alignment structure 50 can include one or more projections on the connecting member 4 and one or more recesses on the inner surface of the pressing tube 3. FIG. 38 is a perspective view of connecting member 4 in pressing tube 3 and shows the alignment structure 50.

The alignment structure 50 can utilize projections and cooperating recesses that are suitably shaped and sized. An embodiment of an alignment structure 50 is shown in FIG. 39, which is cross-sectional view taken along section F-F in FIG. 38. As seen in FIG. 39, the embodiment of the alignment structure 50 includes a projection 51 that projects from a surface of the connecting member 4. The projection 51 cooperatively mates in a recess 52 in the pressing tube 3. One or more projections 51 and/or one or more recesses 52 can be included.

In alternative embodiments, the alignment structure 50 can include one or more projections on the inner surface of the pressing tube 3 and one or more recesses on the connecting member 4.

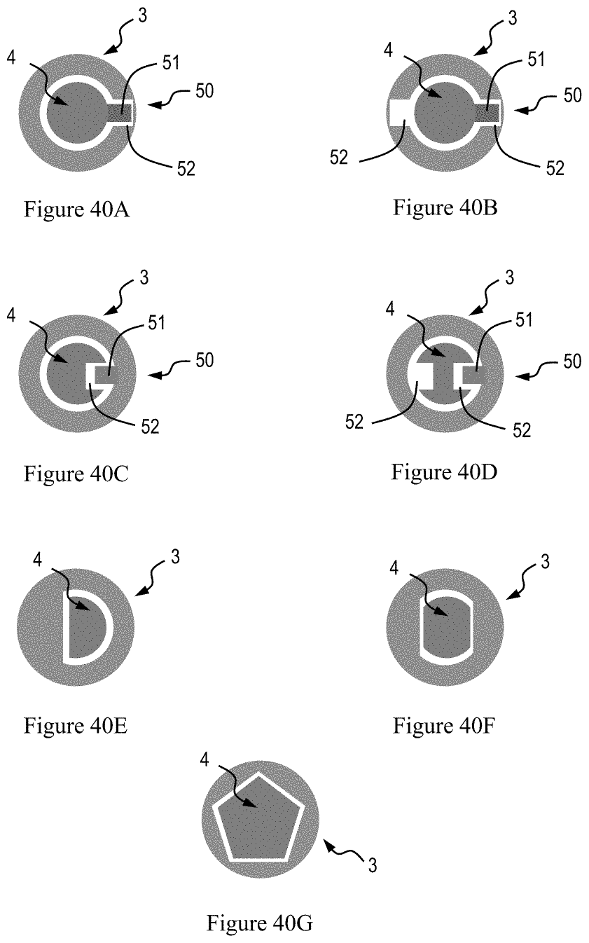

FIGS. 40A to 40G schematically illustrate cross-sections of alternative embodiments of the alignment structure 50. As seen variously in FIGS. 40A to 40D, the alignment structure 50 includes one or more projections 51 on the outer surface of the connecting member 4 and one or more cooperating recesses 52 on the inner surface of the pressing tube 3 (see FIGS. 40A and 40B), or the alignment structure 50 includes one or more projections 51 on the inner surface of the pressing tube 3 and one or more cooperating recesses on the connecting member 4 (see FIGS. 40C and 40D). In still further embodiments, the cross-sectional shape of the connecting member 4 and the cross-sectional shape formed by the inner surfaces of the pressing tube cooperate to prevent rotation of the connecting member 4 relative to the pressing tube 3. Example shapes are shown in FIGS. 40E to 40G and include D-shapes (where one or more outer surfaces of the connecting member 4 are flat and one or more other surfaces of the connecting member 4 have a curvature) (see FIGS. 40E and 40F) and polygonal shapes, such as a pentagon (see FIG. 40G), although other polygon shapes can be used so long as the polygon shape, relative to the shape formed by the interior surfaces of the pressing tube 3, to do not allow for rotation of the connecting member 4 relative to the pressing tube 3.

In still further alternative embodiments, the alignment structure 50 can be located at any suitable location along the length of the connecting member 4. For example, the alignment structure 50 can be located coincident to the proximal end connecting portion 44 (as shown at section F-F in FIG. 38), or coincident to a location along the length of the rod-shaped portion 43 (as shown at section G-G in FIG. 38) or located coincident to the distal end connecting portion 41 (as shown at section H-H in FIG. 38).

### NEW EMBODIMENTS

Although the first embodiment of the present invention has been described above in detail with reference to the drawings, the specific configuration is not limited to this embodiment, and design changes and the like within a range not deviating from the gist of the present invention are also included. In addition, the constituent elements shown in the above-described embodiment and the deformation examples described below can be appropriately combined and configured.

Modification 1: In the above embodiment, the clip 2 has the first arm 211 and the second arm 212, but the mode of the clip 2 is not limited thereto. The clip 2 may have a plurality of arms that can be opened and closed, and may have, for example, four arms.

Modification 2: In the above embodiment, there are two retractable wings 31,311, but the number or retractable wings 31,311 is not limited. The holding tube 3 can incorporate other number of retractable wings 31,311 and may have, for example, one, three or four retractable wings 31,311.

Industrial Applicability: The present invention can be applied to a cartridge or the like for storing a clip unit.

Although the present disclosure has been described in connection with example embodiments thereof, it will be appreciated by those skilled in the art that additions, deletions, modifications, combinations, and substitutions not specifically described may be made without department from the spirit and scope of the disclosure as defined in the appended claims. Thus, it is intended that the present invention cover the additions, deletions, modifications, combinations, and substitutions of this disclosure provided they come within the scope of the appended claims and their equivalents.

What is claimed is:

1. A clip unit, comprising:
a clip having a plurality of arms configured to be opened and closed; and
a connecting member including a rod-shaped portion having a longitudinal axis extending in a longitudinal direction and that longitudinally connects a distal end connecting portion and a proximal end connecting portion,
wherein the distal end connecting portion is releasably connected to the clip and the proximal end connecting portion is configured to be connected to a power transmission member of a clip applicator,
wherein the rod-shaped portion, in a cross-section perpendicular to the longitudinal axis, has a first length in a first direction that is perpendicular to the longitudinal axis and a second length in a second direction that is perpendicular to the longitudinal axis,
wherein the first direction and the second direction are perpendicular to each other, and
wherein the first length is shorter than the second length.

2. The clip unit according to claim 1, wherein a direction of opening and closing of the plurality of arms substantially coincides with the first direction.

3. The clip unit according to claim 1, wherein the plurality of arms are formed of a plate material, and
wherein, when the plurality of arms are in a closed state and connected to the connecting member, a plate thickness direction of the plurality of arms at a location where the plurality of arms are opposed substantially coincides with the first direction.

4. The clip unit according to claim 1, wherein a length of the distal end connecting portion in the first direction defines a third length, and wherein the first length is shorter than the third length.

5. The clip unit according to claim 1, wherein the distal end connecting portion includes a breakage portion which breaks at a predetermined tensile force, and
wherein a breaking strength of the breakage portion is lower than a breaking strength of the rod-shaped portion.

6. The clip unit according to claim 1, further comprising a pressing tube including a pressing tube body and at least one retractable wing,
wherein at least a portion of a proximal end of the clip is translatable into an interior space of the pressing tube body,
wherein the at least one retractable wing is movable between a protruding state and a retracted state, and
wherein, in the protruding state, a body of the at least one retractable wing protrudes outward in a radial direction with respect to an outer peripheral surface of the pressing tube body 30 and, in the retracted state, the body of the at least one retractable wing is located within an envelope of the pressing tube body.

7. The clip unit according to claim 6, wherein, in the retracted state, a minimum separation distance in the first direction between the body of the at least one retractable wing and a surface of the rod-shaped portion defines a fourth length, and
wherein the first length is equal to or less than a fourth length.

8. The clip unit according to claim 7, wherein the first length is equal to the fourth length.

9. The clip unit according to claim 7, wherein, when the at least one retractable wing is in the retracted state and the connecting member translates relative to the pressing tube proximally within the interior space of the pressing tube body, the distal end connecting portion of the connecting member contacts the body of the at least one retractable wing.

10. The clip unit according to claim 6, wherein the at least one retractable wing is a first retractable wing and the pressing tube further includes a second retractable wing, and
wherein the first retractable wing and the second retractable wing are located at radially opposed positions.

11. The clip unit according to claim 10, wherein, in the retracted state, a minimum separation distance in the first direction between the first retractable wing and the second retractable wing defines a fourth length, and
wherein the first length is equal to or less than a fourth length.

12. The clip unit according to claim 11, wherein the first length is equal to the fourth length.

13. A clip applicator, comprising:
a clip having a plurality of arms configured to be opened and closed;
a power transmission member configured to operate the clip;
a connecting member including a distal connecting portion that is releasably connected to the clip, a proximal connecting portion that is releasably connected to the power transmission member, and a rod-shaped portion having a longitudinal axis extending in a longitudinal direction and that connects the distal connecting portion and the proximal connecting portion in a longitudinal direction; and
a sheath through which the clip, the power transmission member, and the connecting member pass,
wherein the rod-shaped portion, in a cross-section perpendicular to the longitudinal axis, has a first length in a first direction that is perpendicular to the longitudinal axis and a second length in a second direction that is perpendicular to the longitudinal axis,
wherein the first direction and the second direction are perpendicular to each other, and
wherein the first length is shorter than the second length.

14. The clip applicator according to claim 13, further comprising a pressing tube including a pressing tube body and at least one retractable wing,
wherein at least a portion of a proximal end of the clip is translatable into an interior space of the pressing tube body,
wherein the at least one retractable wing is movable between a protruding state and a retracted state, and
wherein, in the protruding state, a body of the at least one retractable wing protrudes outward in a radial direction with respect to an outer peripheral surface of the pressing tube body 30 and, in the retracted state, the body of the at least one retractable wing is located within an envelope of the pressing tube body.

15. The clip applicator according to claim 14, wherein, in the retracted state, a minimum separation distance in the first direction between the body of the at least one retractable wing and a surface of the rod-shaped portion defines a fourth length, and
wherein the first length is equal to or less than a fourth length.

16. The clip applicator according to claim 15, wherein the first length is equal to the fourth length.

17. The clip applicator according to claim 15, wherein, when the at least one retractable wing is in the retracted state and the connecting member translates relative to the pressing tube proximally within the interior space of the pressing tube body, the distal end connecting portion of the connecting member contacts the body of the at least one retractable wing.

18. The clip applicator according to claim 14, wherein the at least one retractable wing is a first retractable wing and the pressing tube further includes a second retractable wing, and
wherein the first retractable wing and the second retractable wing are located at radially opposed positions.

19. The clip applicator according to claim 18, wherein, in the retracted state, a minimum separation distance in the first direction between the first retractable wing and the second retractable wing defines a fourth length, and
wherein the first length is equal to or less than a fourth length.

20. The clip applicator according to claim 19, wherein the first length is equal to the fourth length.
